(19) United States
(12) Patent Application Publication
(10) Pub. No.: US 2023/0130458 A1
(43) Pub. Date: Apr. 27, 2023

(54) CATHETER WITH ENERGY DELIVERY MEMBER AND VALVE FOR INTRAVASCULAR LITHOTRIPSY

(71) Applicant: Daniel Ezra Walzman, Teaneck, NJ (US)

(72) Inventor: Daniel Ezra Walzman, Teaneck, NJ (US)

(21) Appl. No.: 18/085,661

(22) Filed: Dec. 21, 2022

Related U.S. Application Data
(63) Continuation-in-part of application No. 16/875,122, filed on May 15, 2020, now Pat. No. 11,564,729.
Application No. 16/875,122 is a continuation-in-part of application No. 16/870,045, filed on May 8, 2020, now Pat. No. 11,006,996.

(51) Int. Cl. A61B 18/14

(52) U.S. Cl. CPC A61B 18/1492; A61B 2018/0022

(57) ABSTRACT

A catheter for intraluminal lithotripsy including a first lumen, an energy delivery member supported by the catheter body. The energy delivery member includes a passageway, a valve positioned in the passageway and an energy emitter configured to communicate energy to target tissue. A method for performing intraluminal lithotripsy is also disclosed.

### BACKGROUND OF THE INVENTION

This application is a continuation in part of application Ser. No. 16/875,122, filed May 15, 2020, which is a continuation in part of application Ser. No. 16/870,045, filed May 8, 2020, now U.S. Pat. No. 11,006,996. The entire contents of each of these applications are incorporated by reference herein.

### FIELD OF THE INVENTION

The present invention relates generally to catheters placed in a minimally invasive manner, and, more particularly, to catheters having expandable structures with energy emitters and valves for intravascular lithotripsy.

### BACKGROUND OF THE RELATED ART

The use of devices in conjunction with medical procedures for controlling blood flow in a blood vessel is taught by the prior art. Among the most common is a balloon catheter. The balloon catheter, such as taught in the prior art, may be used to achieve isolation of a body part from its blood supply as the balloon is inflated (expanded) to occupy the vessel space and block blood flow.

One of the problems associated with using balloons is that although control of the blood flow through a portion of the blood vessel is achieved, including blockage of the blood supply to a targeted site, blood flow is completely interrupted to other sites near the targeted site. This shortcoming can be tolerated for a short duration because when one blood vessel becomes blocked, the body normally increases the blood flow through other, essentially paralleling blood vessels. However, such interruption of blood flow becomes problematic when used for a longer duration. Complex medical procedures may not be achieved during said short duration resulting in injury to other sites or requiring multiple operations at the same targeted site. The need exists for a device for better controlling blood flow during the surgical procedure.

Additionally, current bypass catheters are designed to be surgically implanted, which is not practical for immediate relief of progressive ischemia caused by a sudden blockage of a blood vessel, such as from a thrombus or embolus.

Various devices are known for performing thrombectomy, i.e., removal of a blood clot from a vessel. These include for example mechanical thrombectomy devices with rotational element(s) to break up the clot, devices that deliver thrombolytics to dissolve blood clots, devices that delivery vibrational energy in the form of continuous or pulsating waves, etc. However, these devices do not provide for adequate controlling of blood flow during the procedure. Furthermore, these procedures can often be lengthy, and most often do not provide immediate restoration of flow to the ischemic territory.

It would be advantageous to provide for control of blood flow during removal or treatment of a blood clot or other blockages or removal or softening calcifications. This would be particularly advantageous in procedures of relatively long duration, such as in the use of thrombolytics wherein the blood clot lyses from lytic infusion over time, since it would provide immediate and continuous reperfusion. It is further advantageous to provide immediate restoration of downstream blood flow, allowing time for amelioration of a blockage while stopping further progression of ischemic injury to the involved vascular territory. None of the current devices achieves this.

### SUMMARY OF THE INVENTION

The present invention overcomes the problems and deficiencies of the prior art. The present invention provides an improved catheter and method for use in the vascular system of the body and surmounts the problem of complete blood interruption that causes ischemia, which if not rapidly reversed will result in permanent injury. That is, the present invention is deployed to address a clot or other blockage in an artery or vein that is causing ischemia or heart strain because of the lack of flow through.

Additionally, the present invention provides in some embodiments an improved catheter and method of use for intraluminal lithotripsy, and in certain applications for softening the calcium of a highly calcified valve, such as a cardiac valve.

The present invention provides in some aspects a bypass catheter, placed in the body temporarily, i.e., during the surgical procedure, or for a fixed period of time, having a distal opening (hole) and more proximal intravascular opening (hole) that enables blood flow from a region proximal of the blood clot to a region distal of the clot during the clot treatment procedure. Various embodiments of the bypass catheters are disclosed herein, which include different devices for treating/removing blood clots. In some embodiments, the catheter also includes structure that limits retrograde blood flow through the catheter to enhance the reperfusion function of the catheter. In some embodiments, the catheter includes a filter at a distal portion to capture or block particles.

The present invention in some embodiments includes a temporary bypass balloon mounted catheter, a single lumen difficult access support catheter, and a rotating irrigating and aspirating thrombectomy device. These are disclosed in application Ser. No. 15/732,397 (temporary bypass balloon catheter); and application Ser. Nos. 15/258,877, 15/538,898, and 15/731,478 (rotating separator, irrigator microcatheter for thrombectomy); and other Walzman single-lumen support disclosures, and the present invention in these embodiments provides an improvement thereof.

The devices of the present invention are capable of being positioned so that at least one or more proximal holes, e.g., one or more side holes, of the device is located on one side of said artery or vein clot/blockage and a more distal hole (or holes), e.g., a distal end hole, of the device is located on the other side of the said artery or vein clot/blockage. Once the device is positioned in the desired region in the vessel, a bypass element of the device allows temporary bypass of flow through the catheter, e.g., through the first or distal segment of the catheter as described below.

In some embodiments, in order to prevent backflow of the blood into the catheter, i.e., into a segment (region) of the catheter proximal of the side hole, structure is provided to restrict back flow. Various embodiments of such structure are disclosed herein and include a valve to provide flow in one direction (distal direction), a smaller (reduced) proximal diameter, or attachment to a pressurized fluid line, or a combination of the above. These are discussed in more detail below.

Moreover, in some embodiments, the catheter of the present invention can have an additional lumen extending in the wall, or substantially in the wall, of the intravascular segment of the catheter, in addition to or instead of the lumen for delivering fluid into a balloon for inflation thereof which can extend in the wall, or substantially in the wall, of the catheter, which would deliver fluid into the clot between the side hole and the end hole via at least one perforation that communicates with the inside of the vessel. This would allow delivery of lytics or other such medications into the clot while there is an effective temporary bypass of flow through the catheter, allowing time for the directly applied medication to break up the clot and dissolve the clot while avoiding progressive ischemic tissue injury during the interim time.

In some embodiments, a balloon (or other anchoring structure) is provided on the outer diameter (outer wall) of the catheter, and the catheter can include an additional lumen within the wall, or substantially within the wall, of the intravascular segment of the catheter for inflation and deflation of the balloon.

Moreover, in some embodiments, a mechanical thrombectomy structure can be provided for breaking up the clot such as side loops as described below that can macerate the clot when rotated.

In some embodiments, aspiration can also be applied to the catheter, which can allow aspiration through the side hole and or through the end hole. If aspiration through the end hole only is desired, then the side hole can be withdrawn into a sheath or otherwise covered, as described below, so that the side hole is blocked and there is no aspiration on the side hole and all aspiration forces are on the end hole. Alternatively, an actively controlled valve can be provided to close the side hole.

There is a critical advantage to the devices of the present invention in that they allow rapid restoration of temporary flow of blood through a blockage to avoid ischemic injury, with immediate restoration of a degree of flow beyond a clot. This will allow additional time to remove or dissolve the clot while allowing flow to the at-risk tissue. Additionally, in the case of pulmonary emboli which are large, there is an additional issue of heart strain due to the lack of outflow from the right side of the heart. The temporary bypass catheters described herein can also help relieve such heart strain by allowing outflow from the right heart past said clot when there are large pulmonary emboli in the main pulmonary arteries.

In accordance with one aspect of the present invention, a surgical apparatus (device) for treating a blood clot or other blockage in a vessel of a patient is provided comprising an elongated member, preferably tubular, having an outer wall, a first opening (hole) at a distal portion and a second opening (hole) spaced proximally from the distal hole. The second hole is preferably positioned in a side of the outer wall. A first lumen within the elongated member is provided for blood flow through the proximal second hole, through the first lumen and exiting the distal first end hole to maintain blood flow during treatment of the blood clot. In some embodiments, the first lumen is a single primary central lumen.

At least one perforation can in some embodiments be positioned between the first hole and the second hole. A second lumen within the wall, substantially within the wall or within the primary lumen of the intravascular segment communicates with the at least one perforation. The second lumen forms a channel for injection of fluid through the at least one perforation into the vessel to treat the blood clot, wherein blood flows into the second hole positioned proximal of the blood clot and exits the first hole distal of the blood clot during injection of the fluid to treat the blood clot.

In some embodiments, there is at least one additional third proximal end-hole, which has an external termination device attached, and remains outside the patient's body at all times. Aspiration can optionally be applied to the third proximal end-hole when desired, to remove clot and debris from the vessel.

In some embodiments, the elongated member has at least one energy emitting element positioned thereon, and in some embodiments they are within or substantially within the wall of said elongate member, to emit energy to aid breakdown and removal of the blood clot. In some embodiments, the energy emitting elements are positioned between the first and second holes of the catheter. In some embodiments, the energy emitting elements comprise ultrasound radiating elements to enhance flow or mixing of the fluid (drug) injected from the catheter into or adjacent the blood clot. In some embodiments, the ultrasound emission of the radiating elements is synchronized with timing of delivery of the fluid. In some embodiments, the energy may directly break up larger pieces of clot. In some embodiments, the energy may help break up, soften, and dissolve calcifications and other hardenings. In some embodiments, cooling elements may be present. In some embodiments, heating elements may be present.

In some embodiments, at least one connector is provided to connect the energy emitters to an energy source for application of energy to the blood clot or other blockage to aid treatment, e.g., removal/dissolution of the blood clot. In some embodiments, the at least one connector is configured to connect the apparatus to an ultrasonic energy source. In some embodiments, the energy emitting elements extend within the wall of the catheter. In some embodiments, the energy emitting elements extend on at least a portion of the surface of the catheter. In some embodiments, the energy emitting elements may be incorporated into at least one balloon extending from the catheter. Such embodiments may be of particular use during intravascular lithotripsy of a cardiac valve or intracranial vessel, where prolonged balloon inflation optimal for the optimal contact and treatment time might not be tolerated without a bypass element allowing egress of blood from the heart and perfusion of the cerebral vascular territory, respectively.

The foregoing energy source and energy emitters may also be utilized in the bulging torus balloon, disclosed in U.S. Pat. No. 10,328,246, the entire contents of which are hereby incorporated herein by reference. Such device can be used during valve lithotripsy while allowing egress of blood from the heart through the central hole of the torus balloon, during prolonged balloon inflation for prolonged contact with the valve, or similarly continued blood flow through a vessel during use in a vessel.

In some embodiments, the apparatus includes a rotatable macerator element positioned between the first and second holes, the macerator element rotatable to break up blood clot and other intravascular debris and blockages.

In some embodiments, the apparatus includes a sheath positioned over the elongated member, the elongated member and sheath relatively movable to selectively cover and expose the side hole, wherein covering of the side hole restricts flow of blood through the side hole. In some embodiments, covering of the hole completely blocks flow of fluid through said side hole. Covering of the side hole can also be used to reverse blood flow so it flows proximally through the catheter lumen. Aspiration can be provided in the lumen to aid or effect such reverse flow.

In some embodiments, the apparatus has features to restrict retrograde blood flow within the catheter such as a valve or a reduced diameter region for the first lumen. In some embodiments, attaching pressurized fluid to the catheter at a proximal region can restrict retrograde blood flow within said catheter.

It should be noted that in some embodiments where there is an additional lumen that courses through the intravascular segment of the elongate body, the device divides proximally into multiple lumens with independent outer walls, preferably outside of the patient's body. Preferably, each lumen ends at its proximal end-hole with an independent external termination device, such as a hub with a luer-lock or diaphragm.

In accordance with another aspect of the present invention, a surgical apparatus (device) for treating a blood clot or other blockage in a vessel of a patient is provided comprising an elongated member having an outer wall, a first opening (hole) at a distal portion and a second opening (hole) spaced proximally from the distal opening. The second hole is preferably positioned in a side of the outer wall. A first lumen within the elongated member provides for blood flow through the proximal hole, through the lumen and exiting the distal first hole to maintain blood flow during treatment of the blood clot. The apparatus includes at least one energy emitter for emitting energy to the blood clot and a connector extending through the elongated member to connect the energy emitter to an external energy source, wherein blood flows into the second hole positioned proximal of the blood clot and exits the first hole distal of the blood clot once the bypass segment is positioned across the blockage. In this position, activation of the energy emitter can also be utilized. In this position, infusion of medications can also be utilized.

In some embodiments, the energy emitter is positioned between the first and second holes. In some embodiments, the energy emitter emits ultrasonic energy to the blood clot. In some embodiments, a switch on the apparatus is provided to activate the energy emitters. In some embodiments, a switch external to the apparatus can activate the energy emitters.

In some embodiments, the apparatus further comprises a second lumen for delivering medication to the blood clot for dissolving the blood clot. In some embodiments, this occurs during application of energy, e.g., ultrasonic energy.

In accordance with another aspect of the present invention, a method for treating a blood clot or other blockage in a vessel of a patient is provided comprising the steps of a) inserting into the vessel a device (apparatus) having a first opening (hole) at a distal portion and a second opening (hole) spaced proximally from the distal hole, the second hole positioned in a side of the outer wall; b) positioning the second hole of the device proximal of the blood clot and the first hole of the device distal of the blood clot to thereby enable blood flow through the proximal hole, through the first lumen and exiting the distal hole to maintain blood flow during treatment of the blood clot; and c) during blood flow through the lumen, applying energy to energy emitters carried by the device to apply energy to the blood clot.

In some embodiments, the method further comprises the step of injecting a thrombolytic fluid through one or more perforations in a side wall of the device.

In some embodiments, the method further comprises the step of selectively blocking blood flow through the second hole and aspirating clot through the first hole, via an external aspirator applied to a third hole, i.e., a proximal end hole external to the patient's body.

In some embodiments, the method further comprises the step of injecting a thrombolytic fluid through one or more perforations in a side wall of the device.

In some embodiments, the method further comprises the step of selectively blocking blood flow through the second hole during subsequent aspiration.

In some embodiments, the device has at least one balloon on the external surface of the elongate member overlying said first lumen. In some embodiments, the device further comprises at least one energy emitter on or carried/supported by the balloon for emitting energy.

In some embodiments, the method further comprises the step of using the device as described herein and advancing the device across a valve, inflating the balloon, while blood flows through the first lumen in either direction needed while the balloon is inflated, activating the energy to break up and soften hardenings in and around the valve, deactivating the energy and deflating the balloon.

In some embodiments, the hardenings are calcifications.

In some embodiments, inflation, energy emission, and deflation, are repeated at least two times.

In some embodiments the method includes the step of rotational maceration prior to aspiration.

In accordance with another aspect of the present invention, a catheter is provided having balloon carrying (supporting) or mounting one or more energy emitters for treating blockages. The balloon has a passageway for blood flow. More specifically, the catheter can have a torus balloon for energy delivery and can have a single lumen therein, which can allow passage of a wire, fluid injections, and/or fluid for inflation of the balloon. In other embodiments, the catheter mounted torus balloon for energy delivery can have a single catheter lumen exclusively for the balloon. In other embodiments, the catheter mounted torus balloon for energy delivery may have more than one catheter lumen. There may be a single balloon or multiple balloons. A balloon can be on any segment of the catheter. In some embodiments, the energy emitting elements may extend onto the outer surface of at least one balloon.

In accordance with another aspect of the present invention, a catheter for intraluminal lithotripsy is provided having an outer wall, at least one torus balloon mounted on the outer wall, a first lumen extending therein, at least one energy emitter for emitting energy to break down calcium, mounted on the balloon and a connector connecting the energy emitter to an external energy source, the connector extending through the catheter.

In some embodiments, the catheter is capable of prolonged inflation of the at least one torus balloon within a cardiac valve, without critically obstructing cardiac outflow. In some embodiments, the catheter is capable of prolonged inflation of the at least one torus balloon within a vessel, without critically obstructing blood flow. Thus, the opening in the torus balloon allows blood flow while the balloon is inflated which in the absence of such opening would cut off flow as the inflated balloon fills the vessel lumen. In preferred embodiments, the balloon is inflated so the energy emitters are in contact with the target tissue, e.g., the calcifications in the vessel lumen.

In some embodiments, the catheter includes a second lumen, wherein the first lumen is dedicated solely for the inflation and deflation of the torus balloon.

In some embodiments, the catheter includes an energy emitter. In other embodiments, the catheter includes a plurality of energy emitters spaced apart on the torus balloon. In some embodiments, the at least one energy emitter comprises a plurality of ultrasound radiating elements.

In some embodiments, the torus balloon has an opening to provide passage of blood therethrough. The torus balloon in some embodiments can be eccentrically mounted (e.g., offset from a longitudinal axis of the catheter) so a portion or a majority of the balloon is offset to one side of the longitudinal axis, and the passage in the balloon is parallel to the longitudinal axis of the catheter. In some embodiments, the torus balloon has a channel therein to receive the catheter, the channel radially spaced from the passage.

In some embodiments, the torus balloon has an outer surface extending circumferentially, and a passage of the torus balloon is parallel to a longitudinal axis of the catheter, the at least one energy emitter including a plurality of energy emitters on the circumference of the torus balloon to apply energy radially from the circumference of the balloon.

In some embodiments, the catheter includes a filter positioned distal of the torus balloon to capture particles. The filter can also optionally be provided on the other catheters disclosed herein.

In accordance with another aspect of the present invention, a method of valve lithotripsy is provided including the steps of introducing the foregoing torus balloon across a valve, inflating the torus balloon, emitting energy over a period of time, subsequently stopping emitting energy, deflating the balloon, and removing the catheter.

In some embodiments, the inflation, emitting of energy, and deflation, are repeated at least two times prior to removal of the catheter.

In accordance with another aspect of the present invention, a catheter for intraluminal lithotripsy is provided comprising an outer wall and at least one balloon extending from the outer wall, the balloon having a first portion, a second portion proximal of the first portion and an intermediate portion between the first and second portions. A transverse dimension of the intermediate portion is less than a transverse dimension of the first and second portions. The catheter has a first lumen, at least one energy emitter carried/supported or mounted on the balloon for emitting energy to break down or soften calcium and a connector connecting the at least one energy emitter to an external energy source, the connector extending through the catheter.

In preferred embodiments, the balloon is a torus balloon. In some embodiments, the balloon has figure eight configuration.

In some embodiments, the catheter is capable of prolonged inflation of the balloon within a cardiac valve, without critically obstructing cardiac outflow.

In some embodiments, the at least one energy emitter includes an energy emitter on the second portion of the torus balloon facing the first portion and at least one energy emitter on the first portion of the torus balloon facing the second portion. In some embodiments, the first and second portions are configured to press against opposing sides of the cardiac valve. In some embodiments, the intermediate portion of the balloon forms a waist portion creating a gap between the first and second portions of the torus balloon. The waist portion can be configured for positioning in an orifice of a cardiac valve and the first and second portions of the torus balloon press against opposing sides of the cardiac valve, e.g., press against the sides of the valve leaflets.

In some embodiments, the balloon has an opening to provide a passage for blood therethrough. In some embodiments, the balloon is eccentrically mounted (e.g., offset from a longitudinal axis of the catheter) so a portion, e.g., a majority of the balloon is offset to one side of the longitudinal axis, and the passage is parallel to the longitudinal axis. In some embodiments, the balloon has a channel to receive the catheter, the channel radially spaced from the opening in the balloon.

In some embodiments, the catheter has a filtering member positioned distal of the balloon to capture particles.

In some embodiments, the catheter has an outer wall, a lumen, a first hole at a distal portion positioned distal of the balloon and a second hole spaced proximally from the first hole and positioned proximal of the balloon and positioned in a side of the outer wall, wherein blood flows through the second hole, through the first lumen and exits the first hole while the balloon is inflated and energy is emitted by the at least one energy emitter.

In some embodiments, an axially slidable member is slidable relative to the catheter, the catheter and sliding member relatively movable to selectively cover and expose the second hole, wherein covering of the second hole restricts flow of blood through the second hole. The axially slidable member in some embodiments is external to the catheter; in other embodiments it is internal of the catheter.

In some embodiments, the catheter includes a valve to restrict retrograde blood flow through the elongated member.

In some embodiments, the energy emitter applies ultrasonic energy.

In accordance with another aspect of the present invention, a method for reducing calcium at a cardiac valve of a patient is provided comprising:
a) inserting into the vessel a device having at least one balloon extending from the outer wall, the balloon having a first portion, a second portion proximal of the first portion and an intermediate portion between the first and section portions, a transverse dimension of the intermediate portion being less than a transverse dimension of the first and second portions of the balloon, and at least one energy emitter on the first portion of the balloon and at least one energy emitter on the second portion of the balloon;
b) positioning the balloon adjacent the cardiac valve so the first portion faces a first side of the valve and a second portion faces a second opposing side of the valve and the intermediate portion is positioned in a valve orifice; and
c) applying energy to the at least one energy emitter to apply energy to the first and second sides of the cardiac valve to break down or soften calcium.

In some embodiments, the balloon is a torus balloon and is eccentrically mounted (e.g., offset from a longitudinal axis of the catheter) and has an opening for passage of blood therethrough to pass the cardiac valve that is also positioned eccentrically (e.g., such that the opening is offset from the longitudinal axis of the catheter).

In some embodiments, the balloon is a torus balloon and the catheter has an outer wall, a lumen, a first hole at a distal portion positioned distal of the torus balloon and a second hole spaced proximally from the first hole and positioned proximal of the torus balloon and positioned in a side of the outer wall, wherein blood flows through the second hole, through the first lumen and exits the first hole while the torus balloon is inflated to bypass the cardiac valve. In some embodiments, the balloon is a torus balloon, and the torus balloon is inflated to fill a lumen of the vessel and the first portion presses against the first side of the cardiac valve and the second portion presses against the second side of the cardiac valve, and blood bypasses the inflated balloon as the energy emitters apply energy to the first and second sides of the cardiac valve.

In another aspect of the present disclosure, a catheter is disclosed for intraluminal lithotripsy. The catheter includes a catheter body, an energy delivery member that is supported by the catheter body such that the energy delivery member extends radially outward therefrom and a connector. The catheter body defines a longitudinal axis and includes a first lumen extending therethrough. The energy delivery member includes a body, a passageway that extends through the body in generally parallel relation to the longitudinal axis, a valve that is positioned within the passageway to inhibit blood flow through the energy delivery member and an energy emitter that is configured to communicate energy to target tissue to facilitate treatment thereof. The energy emitter in some embodiments is supported adjacent to an outer surface of the body. The connector extends from the energy emitter to an external energy source to thereby supply energy to the energy emitter.

In some embodiments, the first lumen is configured to receive a supplemental medical device.

In some embodiments, the energy delivery member may be supported by the catheter body such that the passageway is positioned eccentrically relative to the longitudinal axis.

In some embodiments, the energy delivery member may be expandable (e.g., inflatable).

In some embodiments, the energy delivery member may include a proximal portion, a distal portion and an intermediate portion that is positioned between the proximal portion and the distal portion. In some embodiments, upon expansion of the energy delivery member, the proximal portion and the distal portion may each define a first transverse cross-sectional dimension and the intermediate portion may define a second transverse cross-sectional dimension that is less than the first transverse cross-sectional dimension such that the energy delivery member includes a waist defining a gap that is configured to receive the target tissue.

In some embodiments, the energy delivery member may include a generally toroidal (torus) configuration.

In some embodiments, the energy delivery member may be generally cylindrical.

In some embodiments, the energy delivery member may define a proximal end face and a distal end face. In some embodiments, the proximal end face and the distal end face may each have a generally planar configuration. In some embodiments, the energy emitter may be supported adjacent to at least one of the proximal end face and the distal end face of the energy delivery member.

In some embodiments, the energy delivery member may include a deformable material to allow for reconfiguration of the energy delivery member during insertion and removal of the catheter.

In some embodiments, the energy delivery member may include a first energy delivery member and a second energy delivery member.

In some embodiments, the first energy delivery member and the second energy delivery member may be configured as discrete structures that are spaced axially from each other along the longitudinal axis.

In some embodiments, at least one of the first energy delivery member and the second energy delivery member may be movable along the catheter body.

In some embodiments, the catheter is steerable.

In some embodiments, the valve opens intermittently. In some embodiments, a pressure gradient across the valve causes the valve to open. In some embodiments, blood flows through the valve when the valve is open.

In some embodiments, the valve is configured to treat a heart valve.

In another aspect of the present disclosure, a catheter is disclosed for intraluminal lithotripsy. The catheter includes a catheter body, an impeller that is rotatably positioned within the catheter body to direct blood flow therethrough, an energy delivery member extending radially outward therefrom and a connector. The energy delivery member includes a body with a generally annular transverse cross-sectional configuration and can be supported in some embodiments adjacent to an outer surface of the body. The energy emitter is configured to communicate energy to target tissue to facilitate treatment thereof and the connector extends from the energy emitter to an external energy source to thereby supply energy to the energy emitter.

The catheter body defines a longitudinal axis and in some embodiments is configured to receive a supplemental medical device therethrough, or alternatively, thereover.

In some embodiments, the impeller may be configured to selectively direct blood flow through the catheter body in a first (e.g., distal) direction and in a second (e.g., proximal) direction that is opposite (or generally opposite) to the first direction.

In some embodiments, the impeller is positioned within a lumen of the catheter. In some embodiments the impeller is positioned in a passageway of the energy delivery member.

In some embodiments, the catheter body may define a first lumen that is configured to receive the impeller and a second lumen. The second lumen can be configured to receive the supplemental medical device.

In some embodiments, the catheter body and the impeller may be configured such that the impeller rotates about the supplemental medical device upon insertion of the supplemental medical device into the catheter body.

In some embodiments, the energy delivery member may be expandable (e.g., inflatable).

In some embodiments, the energy delivery member may include a proximal portion, a distal portion and an intermediate portion that is positioned between the proximal portion and the distal portion. In some embodiments, upon expansion of the energy delivery member, the proximal portion and the distal portion may each define a first transverse cross-sectional dimension and the intermediate portion may define a second transverse cross-sectional dimension that is less than the first transverse cross-sectional dimension such that the energy delivery member includes a waist defining a gap that is configured to receive the target tissue.

In another aspect of the present disclosure, a method of performing an intraluminal lithotripsy procedure is disclosed that includes inserting a catheter having a catheter body and an energy delivery member that is supported by the catheter body such that the energy delivery member extends radially outward therefrom, wherein the energy delivery member includes a passageway extending therethrough; intermittently inhibiting blood flow through the energy delivery member using a valve supported within the passageway; positioning the energy delivery member adjacent to target tissue; and applying energy to the energy delivery member to treat the target tissue.

In some embodiments, applying energy to the energy delivery member may include communicating energy from an external energy source to an energy emitter that is supported adjacent to or on an outer surface of the energy delivery member.

In some embodiments, positioning the energy delivery member adjacent to the target tissue may include expanding the energy delivery member such that the target tissue is received within a gap that is defined between proximal and distal portions of the energy delivery member.

In some embodiments, positioning the energy delivery member adjacent to the target tissue may include positioning a first energy delivery member distally of the target tissue and positioning a second energy delivery member proximally of the target tissue. In some embodiments, the target tissue is a valve such as a cardiac valve.

In some embodiments, the method may further include directing blood flow through the catheter by rotating an impeller positioned within the catheter body.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a side view of one embodiment of the bypass catheter of the present invention.

FIG. 1A is a transverse cross-sectional view of the catheter of FIG. 1.

FIG. 1B is a transverse cross-sectional view of an alternate embodiment of the catheter of FIG. 1.

FIG. 1C is a transverse cross-sectional view of another alternate embodiment of the catheter of FIG. 1.

FIG. 2 is a side view of an alternate embodiment of the bypass catheter showing in dashed lines the inner diameter of the proximal segment.

FIG. 3 is a side view of an alternate embodiment of the bypass catheter of the present invention shown connected to a pressurized fluid column via the third hole at the proximal end of the catheter.

FIG. 4 is a side view of an alternate embodiment of the bypass catheter of the present invention having perforations for infusion of medication from the catheter into the vessel.

FIG. 4A is a transverse cross-sectional view of the catheter of FIG. 4.

FIG. 4B is a transverse cross-sectional view of an alternate embodiment of the catheter of FIG. 4.

FIG. 4C is a transverse cross-sectional view of another alternate embodiment of the catheter of FIG. 4.

FIG. 5 is side view of an alternate embodiment of the bypass catheter of the present invention.

FIG. 6 is side view of an alternate embodiment of the bypass catheter of the present invention having ultrasonic energy emitters.

FIG. 6A is a transverse cross-sectional view of the catheter of FIG. 6.

FIG. 7 is side view of an alternate embodiment of the bypass catheter of the present invention having a plurality of electrodes.

FIG. 7A is a transverse cross-sectional view of the catheter of FIG. 7.

FIG. 8 is side view of an alternate embodiment of the bypass catheter of the present invention having a rotational mechanical thrombectomy device.

FIG. 9 is side view of an alternate embodiment of the bypass catheter of the present invention having a filter attached to the distal segment.

FIG. 10 is side view of an alternate embodiment of the bypass catheter of the present invention having a filter tethered to the distal end.

FIG. 11 is side view of an alternate embodiment of the bypass catheter of the present invention having a filter and an expandable member (e.g., a balloon) with a plurality of electrodes connected thereon, the expandable member shown in the inflated condition.

FIG. 12 is a side view of an alternate embodiment of a catheter of the present invention having an energy delivery member with a plurality of energy emitters (e.g., electrodes) supported thereby (connected thereto).

FIG. 12A is a side view of an alternate embodiment of the bypass catheter of FIG. 12 in which the energy delivery member includes a valve.

FIG. 12B is a side view of an alternate embodiment of the bypass catheter of FIG. 12A in which the energy delivery member includes a different toroidal (torus) configuration.

FIG. 12C is a side view of an alternate embodiment of the bypass catheter of FIG. 12B.

FIG. 13 is a side view of an alternate embodiment of catheter of the present invention for retrograde flow.

FIG. 14 is a side view of an alternate embodiment of a bypass catheter of the present invention in which the energy delivery member is concentrically mounted and expandable so as to include a waist defining a gap configured to receive target tissue.

FIG. 14A is a side view of an alternate embodiment of the bypass catheter of FIG. 14 which includes a valve positioned therein to inhibit blood flow through the bypass catheter.

FIG. 14B is a side view of an alternate embodiment of the bypass catheter of FIG. 14 in which the energy delivery member is eccentrically-mounted and includes a valve to inhibit blood flow therethrough in a first (e.g., proximal) direction.

FIG. 14C is a side view of an alternate embodiment of the bypass catheter of FIG. 14B in which the valve is configured to inhibit blood flow through the energy delivery member in a second (e.g., distal) direction.

FIG. 14D illustrates a method of treating target tissue (e.g., a calcified heart valve) using the bypass catheter of FIG. 14B.

FIG. 14E is a side view of an alternate embodiment of the bypass catheter of FIG. 14B which includes a different valve.

FIG. 15 is a side view of an alternate embodiment of the bypass catheter of FIG. 14 in which the energy delivery member defines a passageway extending therethrough.

FIG. 15A is a side view of an alternate embodiment of the bypass catheter of FIG. 15 which includes a valve positioned within the passageway.

FIG. 16 is a side view of an alternate embodiment of the bypass catheter of FIG. 14.

FIG. 16A is a side view of an alternate embodiment of the bypass catheter of FIG. 14B which includes a proximal (first) energy delivery member with a valve and a (discrete) distal (second) energy delivery member.

FIG. 17 is a side view of an alternate embodiment of the bypass catheter of FIG. 14 in which the energy delivery member includes a toroidal (torus) configuration.

FIG. 18 is a side view of an alternate embodiment of the bypass catheter of FIG. 17 which includes first and second energy delivery members each having a toroidal (torus) configuration.

FIG. 19 is a side view of an alternate embodiment of the bypass catheter of FIG. 14 in which the energy delivery member includes a (generally) planar (e.g., disc-like) configuration.

FIG. 19A is a side view of an alternate embodiment of the bypass catheter of FIG. 19 in which the energy delivery member is supported by a plurality of braces.

FIG. 20 is a side view of an alternate embodiment of the bypass catheter of FIG. 19 in which the energy delivery member is eccentrically-mounted and includes a valve to inhibit blood flow therethrough.

FIG. 20A is a side view of an alternate embodiment of the bypass catheter of FIG. 20.

FIG. 21 is a side view of an alternate embodiment of the bypass catheter of FIG. 20 which includes a proximal (first) energy delivery member and a (discrete) distal (second) energy delivery member.

FIG. 21A is a side view of an alternate embodiment of the bypass catheter of FIG. 21 in which the proximal energy delivery member is movable via a pusher.

FIG. 21B is a side view of an alternate embodiment of the bypass catheter of FIG. 21A in which each of the proximal and distal energy delivery members are movable via corresponding pushers.

FIG. 22 is a side view of an alternate embodiment of the bypass catheter of FIG. 14 which includes an impeller to direct blood flow through the bypass catheter.

FIG. 22A is a side view of an alternate embodiment of the bypass catheter of FIG. 22.

FIG. 23 is a side view of an alternate embodiment of the bypass catheter of FIG. 12B in which the impeller is incorporated into (supported by) the energy delivery member.

FIG. 24A is a perspective view illustrating the insertion of a delivery (outer) catheter during a surgical procedure.

FIG. 24B is a perspective view illustrating the insertion of an alternate embodiment of the bypass (inner) catheter of FIG. 21 through the delivery catheter of FIG. 24A.

FIG. 24C is a perspective view illustrating deployment of the distal energy delivery member of the catheter of FIG. 24B.

FIG. 24D is a perspective view illustrating retraction of the delivery catheter and the bypass catheter of FIG. 24C.

FIG. 24E is a perspective view illustrating deployment of the proximal energy delivery member of the catheter of FIG. 24D and positioning of the distal energy delivery member adjacent to target tissue (e.g., a calcified valve).

FIG. 24F is a perspective view illustrating positioning of the proximal energy delivery member of the catheter of FIG. 24E adjacent to the target tissue.

FIG. 25 is a schematic representation of an alternate embodiment of the (bypass) catheter of FIG. 14 and shown in a first (initial, normal) configuration.

FIG. 26 is a transverse cross-sectional view of the bypass catheter taken along line 26-26 of FIG. 25.

FIG. 27 is a schematic representation of the bypass catheter of FIG. 25 shown in a second (subsequent, deflected) configuration.

### DETAILED DESCRIPTION OF THE INVENTION

The present invention in some embodiments provides a catheter and method for use in the blood vessel of a patient during a blood clot treatment procedure. The catheter advantageously provides for blood flow during various clot treatment/removal procedures such as mechanical thrombectomy utilizing rotational element(s) to break up the clot, devices that deliver thrombolytics to dissolve blood clots, devices that delivery vibrational energy in the form of continuous or pulsating waves, devices that deliver energy to aid and/or effect blood clot removal, etc., as well as combinations thereof. These various embodiments are described in detail below.

The devices of the present invention provide for controlling blood flow during the procedure to thereby enable immediate and, if desired, continuous, reperfusion during the procedure. They allow for example rapid restoration of temporary flow of blood through a blockage to avoid ischemic injury, with immediate restoration of a degree of flow beyond a clot. This allows additional time to treat, e.g., remove or dissolve the clot or other blockage, while allowing flow to the at-risk tissue.

Additionally, in the case of pulmonary emboli which are large, there is an additional issue of heart strain due to the lack of outflow from the right side of the heart. The temporary bypass catheters described herein can also help relieve such heart strain by allowing outflow from the right heart past said clot when there are large pulmonary emboli in the main pulmonary arteries.

The present invention provides in some embodiments a catheter for use for intraluminal lithotripsy, and in certain applications for softening the calcium of a highly calcified valve, such as a cardiac valve, as described in more detail below. For example, it is envisioned that the catheters described herein may be used pre-operationally to soften any such calcium deposits.

In general, the devices of the present invention achieve such reperfusion by provision of a catheter deployed across a blockage in the vessel. The catheter in some embodiments is a bypass catheter having a distal opening and at least one proximal intravascular opening providing a bypass window, the openings positioned within vessel(s) on either side of the blood clot to be treated as the catheter is positioned across the blockage in the vessel. This enables blood flow from a region proximal of the clot to a region distal of the clot. In some embodiments, the catheter includes additional structure or features that limit retrograde blood to enhance the reperfusion function of the catheter. These various structures/features are discussed in detail below.

In some embodiments, the present invention utilizes in an improvement thereof elements of a temporary bypass catheter and balloon, a single lumen support catheter, and a rotating irrigating and aspirating thrombectomy device.

In some embodiments the device may further comprise a semipermeable filter attached circumferentially at or near its distal end to minimize the risk of emboli during the procedure. The filter can be self-expanding. The filter may have various modalities, to constrain and deploy it as desired. In some embodiments, the filter can be attached to a wire that extends through the entire lumen of the device and deploys distally within the vessel. In some embodiments, the filter is distal to the distal end hole and is tethered to the catheter.

Referring now to the drawings and particular embodiments of the present disclosure, wherein like reference numerals identify similar structural features of the devices disclosed herein throughout the several views, there are illustrated several embodiments of the catheters of the present invention.

Note as used herein, the term “proximal” and “distal” refer to the direction of blood flow with blood flowing in a proximal to distal direction. Also note the terms “apparatus” and “device” and “catheter” are used interchangeably herein. Also note the terms “hole” and “opening” are used interchangeably herein.

“Blood clot treatment” as used herein includes any type of treatment of the blood clot which can include partial removal of the clot, reduction in size of the clot, complete removal of the clot, removal by mechanical thrombectomy, dissolution by medication, etc. The devices of the present invention can also be used for other vascular treatment including removal of other intravascular debris and blockages as well. Thus, the terms “blood blockage treatment” or “vessel blockage treatment” as used herein include blockage due to clots or other blockages.

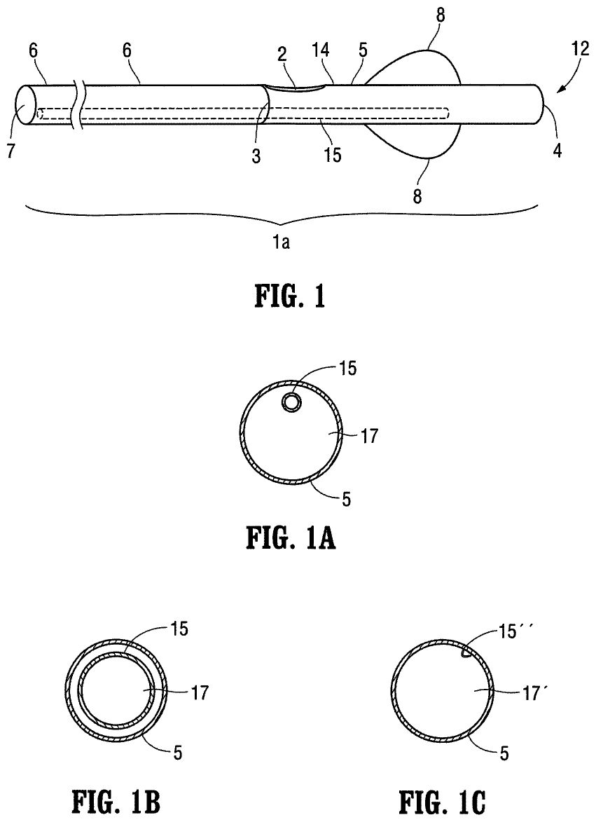

Referring now to FIG. 1, a first embodiment of the bypass catheter of the present invention is illustrated. Note the catheters disclosed herein are also referred to as a device or apparatus. The catheter, designated generally by reference numeral (1a), is in the form of an elongated member, preferably tubular, and has a proximal (end) hole (opening) (7), which in some embodiments is attached to an external termination device, a distal end hole (4) at a distal portion and a side hole (bypass window) (2) disposed upon the outer diameter, i.e., in the wall (14) of the device (1) at the juncture of first (distal) segment (5) and second (proximal) segment (6). Note the segments (5) and (6) indicate the two regions or portions of the catheter (1a) as the catheter (1a) can be an integral tubular structure as shown. However, alternatively, the segments (5) and (6) can be composed of separate elongated tubular members that are attached/joined together. Side hole (2) defines the end of second segment (6) or is at a proximal end of segment (5) and blood flows through side hole (2) and exits through distal hole (4). The outer diameter of first segment (5) and second segment (6) are the same in the illustrated embodiments. However, in alternate embodiments of the catheters disclosed herein, the outer diameter of segment (5) can be greater or less than the outer diameter of segment (6) and one or both of the segments can have a taper. Also, although one side hole is shown throughout the drawings of the various embodiments, it is also contemplated that more than one side hole for blood inflow can be provided in the bypass catheters disclosed herein. Similarly, multiple egress holes can be provided; however, when there is no intervening vascular branching a single end hole is preferred to maximize laminar flow, minimize turbulence, and maximize flow volume and rate.

The bypass catheter (1a) is introduced through an incision in a patient's vessel, most often percutaneously, and often directed through the vasculature to a target site by use of standard endovascular techniques, with the aid of wires, e.g., guidewires, and/or delivery catheters, often under fluoroscopic guidance. The catheter can be inserted over a guidewire extending through proximal opening (7) and lumen (17) of the catheter (1a) and out distal opening (4). While the lumen 17 is illustrated as including a circular (or generally circular) transverse cross-sectional configuration (e.g., diameter), alternative configurations are also contemplated herein. For example, it is envisioned that the lumen 17 may include a non-circular transverse cross-sectional configuration (e.g., square, rectangular, hexagonal, octagonal, pentagonal, a “house” silhouette, oval, elliptical, star-shaped, etc.). In the context of a star-shaped transverse cross-sectional configuration, any style of star may be used including, for example, a six-pointed star, a “Star of David,” etc.

First or distal segment (5) in some embodiments has structure for anchoring device (1a) within the vessel so as to position and maintain side hole (2) at the desired location. This structure can include for example expandable wires which expand to at least the size of the inner diameter of the vessel to hold the device (1a) in place. Alternatively, an expandable balloon can be provided such as balloon (8) shown in FIG. 1, which is attached to first segment (5). The balloon (8) can also serve to regulate flow, and thereby help control contact of any delivered medication with any clot. The balloon (8) is inserted in a deflated collapsed position. Upon inflation via injection of fluid (liquid or gas) through a channel (15) in catheter (1a) which communicates with the interior of balloon (8), the balloon (8) expands from a collapsed condition to an expanded position to at least the inner diameter of the vessel to thereby anchor catheter (1a) in the desired position. Note the catheter (1a) can include a separate channel or lumen (15) (see FIG. 1A) for injection of inflation fluid, e.g., saline, to expand the balloon (8) or for passage of a wire or other elongated mechanism for expanding the wires in the embodiment wherein a mechanical expander is used instead of a balloon for anchoring the catheter (1a). In a preferred embodiment, the additional lumen is within or substantially within the wall of the catheter, thereby minimizing any obstruction within the primary central lumen, and maximizing blood flow through the bypass segment. The term substantially within (or substantially embedded in) the wall as defined herein means more than 75% of the lumen is within the wall either radially with respect to the wall or longitudinally along the length. The anchoring device, e.g., balloon (8), is shown positioned between side hole (2) and distal hole (4), but alternatively, can be positioned in other regions of the catheter, e.g., proximal of side hole (2) in second (proximal) segment (6). Note that anchoring structures can be provided on the other bypass catheters disclosed herein. In some embodiments, no anchor is provided.

While the lumen 15 is illustrated as including a circular (or generally circular) transverse cross-sectional configuration (e.g., diameter), alternative configurations are also contemplated herein. For example, it is envisioned that the lumen 15 may include a non-circular transverse cross-sectional configuration (e.g., square, rectangular, hexagonal, octagonal, pentagonal, a “house” silhouette, oval, elliptical, star-shaped, etc.). In the context of a star-shaped transverse cross-sectional configuration, any style of star may be used including, for example, a six-pointed star, a “Star of David,” etc.

As shown in FIG. 1A, the lumen 15 is positioned within the central lumen 17. Alternatively, the lumen 15′ (FIG. 1B), serving the same function as lumen 15, can be embedded or substantially embedded in the wall of the catheter segment 5. The primary central lumen for blood flow is designated by reference numeral 17′. Alternatively, the lumen 15″ (FIG. 1C), serving the same function as lumen 15, can be adjacent the wall of the catheter segment 5 so it shares a wall of catheter segment 5. Primary central lumen for blood flow is designated by reference numeral 17″.

The device 1a of the present invention is positioned such that side hole (2) is positioned to accept blood flow from the patient and direct the blood through the lumen (17) in the first segment (5) and out through distal hole (4), bypassing said blood flow past a blockage. As noted above, one side hole (2) is illustrated, however, it is also contemplated that more than one side hole (2) can be provided in catheter 1 a, as well as in the other catheters described herein, to provide more than one entry passage for blood flow into the catheter at a region proximal of the vessel blockage.

In some embodiments, there is at least one additional third proximal end-hole, which has an external termination device attached, and remains outside the patient's body at all times. Aspiration from an external aspiration device can optionally be applied to the third proximal end-hole when desired, to remove clot and debris from the vessel.

In some embodiments, an additional lumen is positioned within or substantially within the wall of the catheter and can take a spiral or corkscrew course within the wall to get to (extend to) the balloon, thereby potentially improving the flexibility of the catheter. Similarly, any wiring within the device, e.g., within the wall or substantially within the wall, to transmit energy as in the embodiments described below, when present, can in some embodiments take a similar spiral or corkscrew course as well within the wall. Alternatively, in some embodiments, the additional lumen(s) for delivery of fluids such as medication to the perforations and/or for inflating and deflating the balloon may course freely entirely through the intravascular portion of the single primary central lumen, except for attachments proximal to said perforations and balloon; in effect additional microcatheters coursing through the outer catheter, and attached only distally.

It should be noted that in some embodiments where there is an additional lumen that courses through the intravascular segment of the elongate body, the device can divide proximally into multiple lumens with independent outer walls, preferably outside of the patient's body. Preferably, each lumen ends at its proximal end-hole with an independent external termination device, such as a hub with a luer-lock or diaphragm.

The catheters of the present invention can include structure or features to prevent backflow of blood through the lumen 17. Three alternatives are discussed below which can be used independently or in combination or one or more and can be used with any of the embodiments of the bypass catheters disclosed herein.

FIG. 1 illustrates an embodiment employing valve (3) disposed within the primary central lumen at the juncture of second section (6) with side hole (2). The valve (3) can take various forms such as a leaf valve, flapper valve, etc. The valve can be configured to allow blood flow in one direction, i.e., a distal direction, which prevents flow in a proximal direction without any clinician intervention. Alternatively, the valve can be configured to be opened by intervention of a clinician. In the embodiment of FIG. 1 for example, once the device (1) is positioned in the desired position adjacent the blood clot to be treated, e.g., removed, valve (3) is closed by the user (clinician) to prevent blood entering side hole (2) from flowing back into said second segment (6). The valve can be controlled at a proximal region of the catheter (1), with the control attached by a wire or other elongated member to the valve. By closing the valve (3), the blood is thereby directed through first segment (5), through lumen (17) of segment (5) and out distal end hole (4), and allowed to perfuse the at-risk tissue. Alternatively, the valve can be in a default closed position and opened when a wire is passed through it, subsequently automatically closing when the wire is removed.

During use of the device (1), the distal segment (5) is placed across the target tissue (e.g., a clot, a calcified heart valve, etc.) and blood is then able to bypass the target tissue (depending on the direction of blood flow) through the distal segment (5). For example, it is envisioned that blood may flow through the side hole (2), through the distal segment (5), and out of the distal end hole (4), or that blood may flow through the distal end hole (4), through the distal segment (5), and out of the side hole (2). The device (1) is configured such that the proximal end hole (7) is positioned externally of the patient (e.g., to allow for the aspiration of debris, clot material, etc., therethrough), whereby the proximal segment (6) does not service the bypass procedure.

In certain methods of use, if the side hole (2) is covered (e.g., by patient's tissue), the bypass will be closed, and blood will not flow through the device (1). To establish blood flow, however, it is envisioned that blood may be back bled or actively aspirated out of the body (e.g., via the proximal end hole (7)).

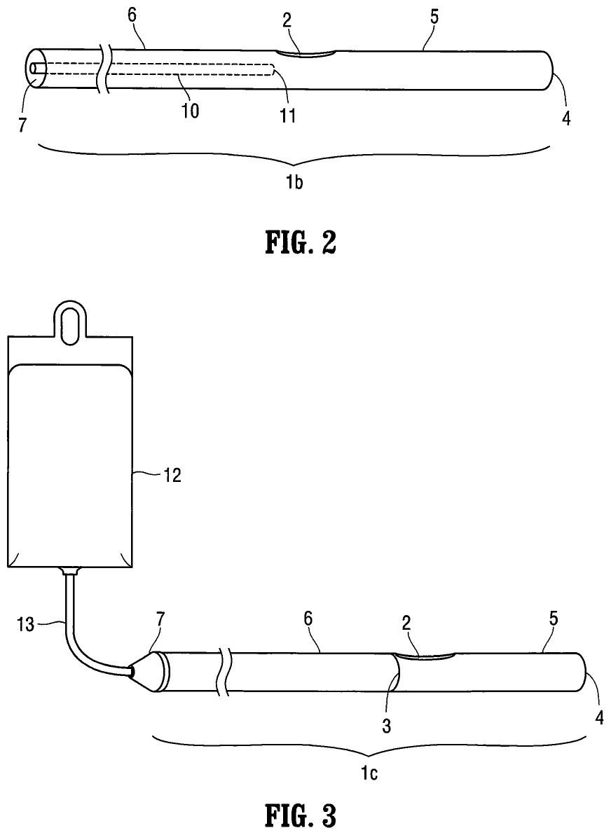

In an alternate embodiment depicted in FIG. 2, instead of a valve, the inner diameter (10) of second segment (6) is less than the inner diameter of first segment (5). That is, the inner wall of second segment (6) is thicker to provide a smaller diameter lumen as compared to the lumen of segment (5) or the second segment (5) can have a smaller diameter forming a smaller lumen. Inner diameter (10) terminates at inner hole (11). Inner hole (11) as shown is smaller than distal end hole (4). The differential of inner diameters acts to constrict backflow and direct blood through first segment (5) to and out end hole (4). In all other respects the catheter (1b) of FIG. 2 is the same as catheter (1a) of FIG. 1 and can optionally include an anchoring structure such as balloon (8) and a valve, but in preferred embodiments does not have a valve.

In some embodiments, both a valve (3) and a reduced inner diameter (10) and inner hole (11) are employed to constrict backflow of blood. As noted above, the valve can be configured to allow blood flow in one direction in its natural state or alternatively the valve can be manipulated by the clinician between an open position to allow blood flow, and a closed position to restrict blood flow when desired.

In a still further embodiment depicted in FIG. 3, pressurized fluid may be introduced into second segment (6) to prevent the backflow of blood. FIG. 3 depicts device (1c) connected to pressurized fluid bag (12) interfacing with proximal end hole (7) via tubing (13), appropriate connectors, and an external termination device at the proximal end hole. Other sources of pressurized fluid are also contemplated such as an injection device. Proximal end hole (7) communicates through second segment (6) to first segment (5) via a lumen extending therein. The pressurized fluid bag (12) may be connected to a flow regulator which is outside the patient's body to allow the user of the device to control flow of fluid through the second segment (6). A pressure gauge can also be provided to regulate the pressure of the fluid delivered through the catheter. Like the catheter (1a) of FIG. 1, the catheter (1c) of FIG, 3 has a side hole (2) for entry of blood to bypass the blockage and distal hole (4) as described above.

The pressurized fluid may be used alone or in conjunction with valve (3) as shown in FIG. 3 and/or in conjunction with reduced diameter inner hole (11) to prevent backflow of blood through the segment (6). Stated another way, pressurized fluid, valve (3) and differential inner diameter (10) and inner hole (11) may all be used concurrently or only one or only two of these features can be used in the catheters disclosed herein. In some embodiments, the outer diameter of the proximal segment (6) and the distal segment 5 may vary as well. This may be particularly useful to limit the sheath size needed to introduce a larger diameter distal segment (6) to a lesion, when used in conjunction with an expandable sheath such as the e-sheath made by Edwards Lifescience.

In some embodiments, a balloon on the catheter or sheath (described below) can be provided which can be selectively inflated if there is a desire to arrest flow and or reverse flow during the clot treatment process, e.g., maceration process, to prevent showering of clots, or to aspirate clots and debris.

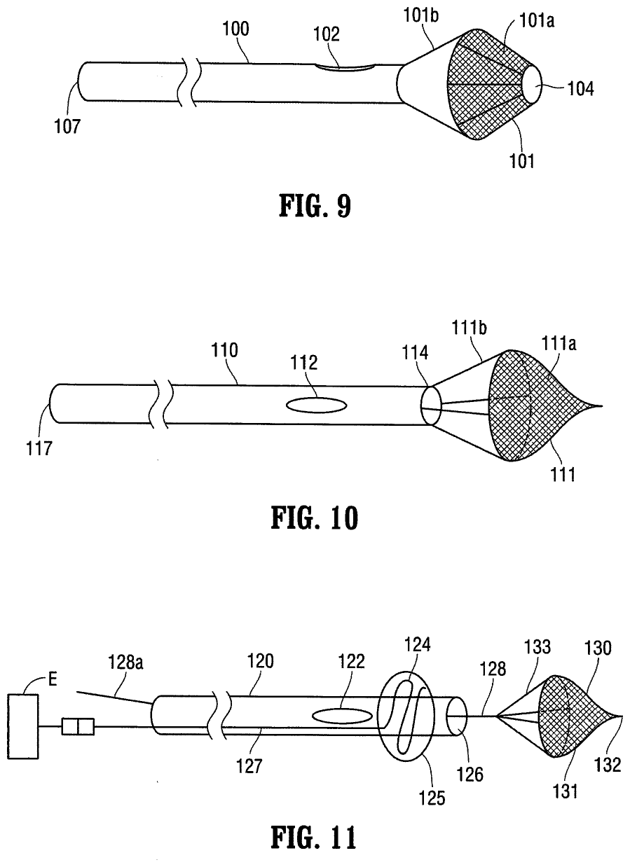

In some embodiments, the catheters can have a filter or distal protection device at a distal portion. FIGS. 9-11 illustrate three embodiments of such filter, mounted in different ways. Note these filters can be utilized with any of the embodiments of the devices (catheters) disclosed herein and FIGS. 9-11 illustrate some examples of such catheters.

In the embodiment of FIG. 9, filter 101 is attached to the distal end of catheter 100, terminating at end hole 104. Catheter 100, like the other catheters disclosed herein, has a proximal opening 107, a side hole 102 for blood inflow (like side hole 2 of FIG. 1) and a distal end hole 104 for blood exit in the bypass manner disclosed herein. In the embodiment of FIG. 10, filter 111 is tethered to the distal end of catheter 110 so it is positioned distal of distal end hole 114. Wires 111b are attached to a distal region of the catheter 110, and extend distally thereof. Catheter 110, like the other catheters disclosed herein, has a proximal opening 117, a side hole 112 for blood inflow (like side hole 2) and a distal end hole 114 for blood exit in the bypass manner disclosed herein. The embodiment of FIG. 11 illustrates a distal filter 130 utilized with a catheter with energy emitters. FIG. 11 is discussed in more detail below.

During use of the catheters 100, 110, the distal regions thereof are placed across the target tissue (e.g., a clot, a calcified heart valve, etc.) and blood is then able to bypass the target tissue (depending on the direction of blood flow) through the distal regions. For example, it is envisioned that blood may flow through the side holes 102, 112, through the distal regions, and out of the distal end holes 104, 114, or that blood may flow through the distal end holes 104, 114, through the distal regions, and out of the side holes 102, 112. The catheters 100, 110 may be configured such that the respective proximal openings 107, 117 are positioned externally of the patient (e.g., to allow for the aspiration of debris, clot material, etc., therethrough), whereby the proximal regions of the catheters 100, 110 do not service the bypass procedure.

In certain methods of use, if the respective side holes 102, 112 are covered (e.g., by the patient's tissue), the bypass will be closed, and blood will not flow through the catheters 100, 110. To establish blood flow, however, blood may be back bled or actively aspirated out of the body (e.g., via the respective proximal openings 107, 117).

Filters 101, 111 and 130 can have a wire, mesh, braid or other filtering material 101a, 111a, 131, respectively, to block/capture particles from traveling downstream in the vessel, while enabling blood flow therethrough. A plurality of wires 101b, 111b, 133 are expandable to move the filter 101, 111, 130, respectively, from a collapsed condition for delivery distal of the blockage to an expanded position. The filter can be self-expandable or can be manually controlled by a wire connected to wires 101b, 111b, 133 and selectively actuable at a proximal region of the catheter outside the patient's body.

The semipermeable filter is attached circumferentially at or near the distal end of the catheter to minimize the risk of emboli during the procedure. The filter may have various modalities, to constrain and deploy it as desired. In some embodiments, the filter can be attached to a wire that extends through the entire lumen of the device and deploys distally within the vessel. The filter can be configured as shown or be of other shapes/configurations.

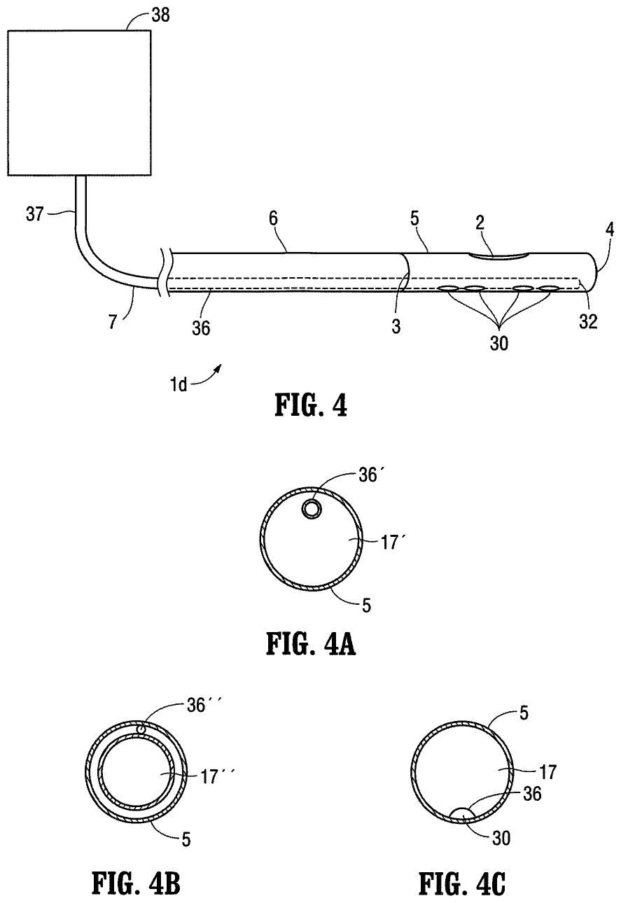

FIG. 4 illustrates an alternate embodiment of the catheter of the present invention wherein first segment (5) is perforated with at least one perforation (30). Perforations (30) of bypass catheter (1d) are end holes, i.e., exit holes, communicating with a separate lumen or channel (36) (FIG. 4A) within catheter (1d). Channel (36) provides an independent irrigation (fluid) channel extending to proximal end hole (7) and in communication with a controller (38) via tubing (37) for controlling the fluid flow through the channel (30). The fluid is introduced into the separate channel (36), flows through the channel (36) extending through segments (5) and (6) and exits perforations (30) to flow into the vessel and particularly the blood clot to dissolve vessel-clogging material of the clot. For example, the fluid may be a medication, for example a lytic such as Alteplase, which dissolves blood clots. The controller (38) is capable of controlling/regulating the flow of medication from controller through lumen (36) and out perforations (30) to effect the dissolution of clots near first segment (5). Alternatively, other methods such as hand injections via a syringe may be employed. The medication has the capability of softening and/or changing the chemical makeup of blood clots adjacent perforations (30) for purposes of dislocating and/or dissolving the clot(s) or other blockage. To enhance dissolution an energy source can be provided which is described in detail below in conjunction with FIG. 6.

While the lumens of the catheters disclosed herein are illustrated as including a circular (or generally circular) transverse cross-sectional configuration (e.g., diameter), alternative configurations are also contemplated herein. For example, it is envisioned that the lumen 36 may include a non-circular transverse cross-sectional configuration (e.g., square, rectangular, hexagonal, octagonal, pentagonal, a “house” silhouette, oval, elliptical, star-shaped, etc.). In the context of a star-shaped transverse cross-sectional configuration, any style of star may be used including, for example, a six-pointed star, a “Star of David,” etc.

One or more of the perforations (30) can be provided between the side hole 2 and distal hole 4 of the bypass catheter 1. Alternatively or additionally, one or more perforations (30) can be provided proximal of the side hole 2 as shown in FIG. 4. Fluid exiting from perforations (30) can affect proximal regions of the blood clot. Note in FIG. 4 the side hole (2) is shown in a middle (intermediate) region of segment (5), however, it should be appreciated that the side hole (2) can be provided along other regions of catheter (id), such as in a proximal region of segment (5) as in the embodiment of FIG. 1. A valve 3 or other flow restricting structure can be provided.

The device (1d) of FIG. 4 can be composed of concentric lumens wherein channel (36) for medication flow communicating with perforations (30) is centered within the lumen (17). In alternate embodiments, the channel is positioned within the primary lumen but off-center, and substantially along the wall of the catheter. In another embodiment shown in FIG. 4B, the fluid delivery lumen leading to the perforations courses substantially through the wall of the intravascular segment of the catheter. More specifically, in FIG. 4C, channel 36 is shown adjacent the wall of the catheter segment 5 so it has a common wall with the catheter. In the alternate embodiment of FIG. 4A, the channel 36′ which functions like channel 36, is positioned off center within the primary central lumen 17′ of catheter segment 5 which functions like lumen 17. In the embodiment, of FIG. 4C, the channel 36″ which functions like channel 36, is embedded in the wall of the catheter segment 5. The primary central lumen which functions like lumen 17 is designated by reference numeral 17″.

In some embodiments, perforations (30) communicate with the area between the internal surface of the outer lumen and the outer surface of the inner lumen 36, the gap extending from perforations (30) to proximal end hole (7) and communicating with the controller (38). This allows medication to be pumped from the controller (38) through the area between the internal surface of the outer lumen and the outer surface of the inner lumen and out perforations (30) to allow the infusion of medication to soften, lyse, or alter the composition of clots or blockages. In the preferred embodiment, the inner channel (or area between the internal surface of the outer lumen and the outer surface of the inner lumen) terminates at the most distal perforation (30) at end (32). Alternatively, the inner channel may terminate in the first segment (5) at or near the end hole (4). In alternative embodiments, there may be one or more slits along the catheter surface, instead of or in addition to said perforations, through which said fluid medication is delivered.

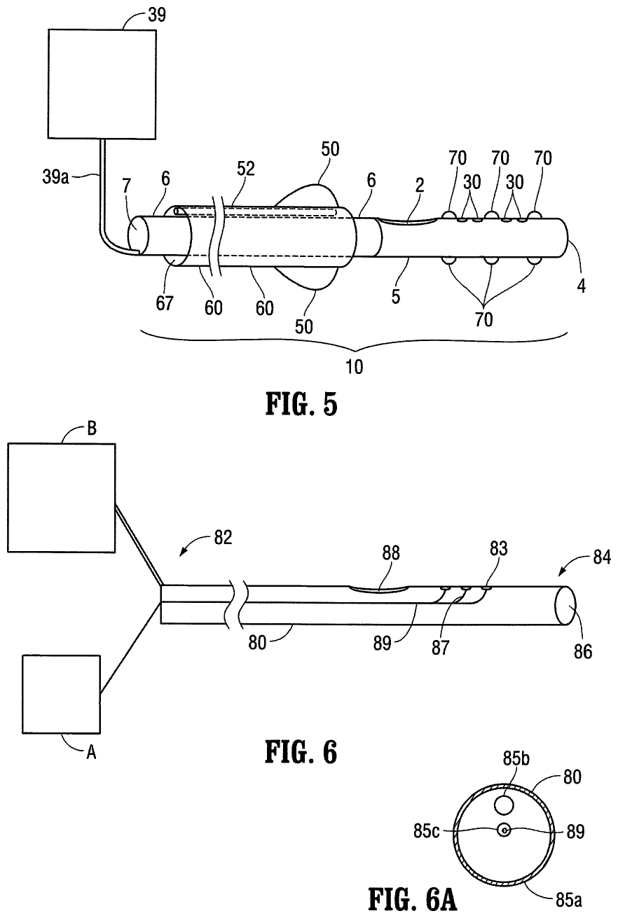

Referring now to FIG. 5, an alternate embodiment of the device of the present invention further includes rotating, macerating and irrigating elements. More particularly, bypass catheter 10 includes a slidable outer support sheath (60) having a proximal opening (67), macerating elements or loops (70), and/or perforations (30) used as irrigating elements to enable outflow of fluid such as medication for dissolving blood clots. The slidable outer support sheath (60) provides a hole covering member and is capable of snugly closing side hole (2) when first segment (5) is withdrawn (moved proximally) inside of said sheath (60) or sheath 60 is advanced (moved distally) to cover side hole (2) or both sheath 60 is moved distally and first segment (5) is moved proximally. Each of these variations can be considered relative movement. In any of these methods, such relative movement is utilized to effect opening (exposure) of side hole (2) and closing (covering) of side hole 2 as desired by the clinician. Movement of the sheath (60) is controlled at a proximal end and movement of the first segment (5) is controlled by movement of the catheter 10 also controlled at the proximal end. When side hole (2) is closed, aspiration of intravascular contents via end hole (4) can be accomplished by applying external aspiration at proximal end hole (7). Note in alternate embodiments, a sheath to cover the side hole can be positioned within the catheter rather than external of the catheter. Such external or internal sheath can be used with any of the embodiments disclosed herein.

Macerating elements (70) extend radially from the catheter (10) and are preferably positioned between the side hole (2) and distal hole (4). Macerating elements (70) macerate the clot as the catheter 10 is rotated to rotate the elements (70). Such rotating can occur concurrently with infusion of medication through perforations (30) to also aid mixing or movement of the medication. Although shown in the form of loops (70), other macerating structure is also contemplated. In an alternate embodiment shown in FIG. 8, instead of the macerating element(s) rotatable by rotation of the catheter, the macerating element is rotated independent of the catheter. As shown in FIG. 8, the macerating element 74, in the form of a wire, extends radially from the bypass catheter 72 and is mounted on a rotating shaft 75. Shaft 75 extends through lumen 77 and can be manually rotated or alternatively rotated by a motor 73, positioned within the catheter or alternatively outside the catheter, to break up the clot. During maceration, blood flows through side hole 78, through lumen 77 and exits distal and hole 76 to bypass the blood clot. Perforations like perforations 30 of FIG. 5 can be provided for fluid, e.g., medication injection. A sliding member, e.g., a sheath, can be provided to selectively cover side hole 78.

While the lumen 77 is illustrated as including a circular (or generally circular) transverse cross-sectional configuration (e.g., diameter), alternative configurations are also contemplated herein. For example, it is envisioned that the lumen 77 may include a non-circular transverse cross-sectional configuration (e.g., square, rectangular, hexagonal, octagonal, pentagonal, a “house” silhouette, oval, elliptical, star-shaped, etc.). In the context of a star-shaped transverse cross-sectional configuration, any style of star may be used including, for example, a six-pointed star, a “Star of David,” etc.

Turning back to FIG. 5, catheter (10) further includes an aspiration controller (39) communicating with proximal end hole (7) via tubing (39a). Such aspiration is utilized to effect backflow of blood through the catheter (10). More specifically, when the side hole (2) is uncovered by sheath (60), the catheter performs its bypass function with blood flowing through the side hole (2) and out the distal end hole 4 to bypass the vessel blockage in the same manner has side hole 2 and distal hole 4 of the aforedescribed embodiments. When side hole (2) is covered by the outer support sheath (60), and the aspiration is activated via controller (39), this results in changing the blood-flow bypass from side hole (2) through distal end hole (4) to instead redirect the blood flow from distal end hole (4) out proximal end hole (7) due to aspiration controller (39) communicating with proximal end hole (7).

Device (10) can also include a backflow valve like valve (3) or other reverse flow restricting features/structure described herein. A balloon (50) can be provided, expandable by inflation fluid injected through a channel (52) in the catheter (10), to expand to a diameter equal to or slightly greater than the internal diameter of the vessel to provide an anchoring force to secure the catheter in position and/or to control flow in the vessel. Note the balloon (5) in the illustrated embodiments is proximal of the side hole (2) but can be positioned in other locations. Other mechanical anchoring elements can alternatively be provided as described above.

If the operator chooses to aspirate from distal end hole (4), the bypass catheter (10) can be pulled back (or the sheath (60) moved forward or both moved relative to each other) so that the side hole (2) is temporarily positioned within sheath (60), which is sized for a snug fit around bypass catheter (10), and aspiration force applied at proximal hole (7) will be transmitted to proximal end hole (4), provided valve (3), when provided, is open during said aspiration. It should be noted that for optimal use of this embodiment of the present invention, first segment (5) fits snugly inside slidable outer support sheath (60) or at least has a minimum gap so inflow of blood through side hole 2 is inhibited or completely restricted when sheath (60) is covering side hole (2).

It should be appreciated that in alternate embodiments, to close off the side hole (2), the hole covering member can be an inner member slidably disposed within the lumen of device (10) and can be moved distally, or the device 10 retracted proximally, or both moved relative to each other, so that the outer wall of the inner member covers the side hole 6, preferably sufficiently tight to reduce or close a gap between the outer wall of the inner member and the inner wall of the device 10 to restrict blood flow therein.

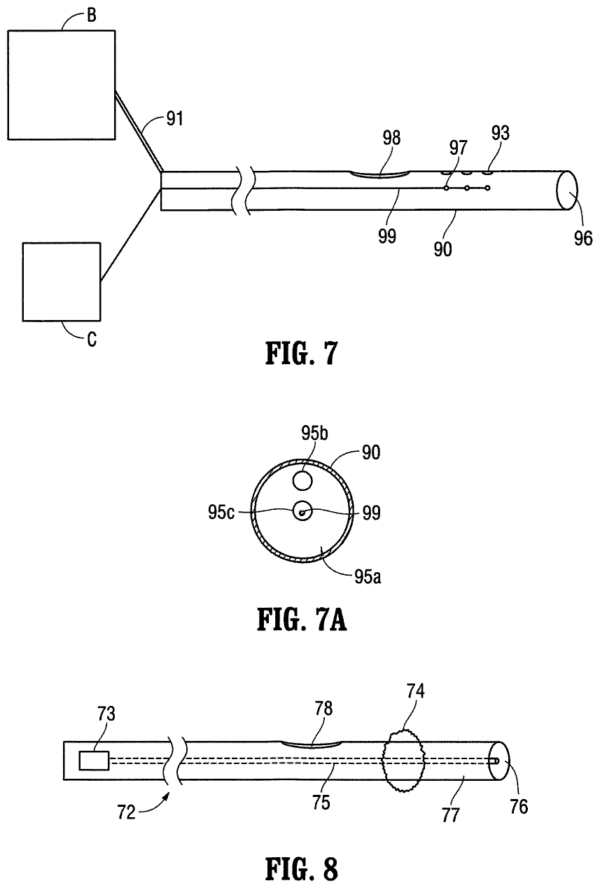

FIGS. 6-7A illustrate alternate embodiments of the bypass catheter of the present invention wherein energy is applied to treat the blood clot, e.g., break up or dissolve the clot. The energy can be used in conjunction with clot dissolution drugs or alternatively used without such drugs relying on mechanical breakup of the blood clot. In some embodiments, ultrasound waves are transmitted. Various frequencies of ultrasound can be utilized. Some frequencies are optimized for clot dissolution, some frequencies are optimized for medication delivery into a clot, some frequencies are optimized for softening calcium, some frequencies are optimized for dissolving calcium, some frequencies are optimized for breaking up calcium and some frequencies are optimized for other uses.

Turning first to the device of FIG. 6, device (bypass catheter) 80 has a proximal end 82, a distal end 84 terminating in a distal exit (end) hole 86 and a side hole 88 in the outer wall of device 80. The bypass catheter 80 can be considered to have two segments or portions, either integral or separate joined components, as described above. The bypass catheter 80 in this embodiment has three channels (lumens): i) main channel 85a which enables blood entering through side hole 88 in the wall of the catheter 80 to flow out distal hole 86 to bypass the blood clot to provide for immediate perfusion, ii) channel 85b for injection of medication such as thrombolytics from fluid source B to dissolve the clot; and iii) channel 85c to contain the wire(s) 89 connecting the ultrasonic source A to the energy emitters (radiating elements) 87 disposed on, e.g., along, the catheter 80. Note that these three lumens 85a, 85b, 85c are provided by way of example since in alternate embodiments the wires 89 can be positioned in the fluid channel 85b in which case the catheter 80 would have two lumens instead of three. Alternatively, the wires 89 can be embedded or substantially embedded in a wall of the catheter 80. In a preferred embodiment, both the wires and the additional lumen for fluid/medication delivery are both embedded fully or substantially within the wall of the intravascular segment of the catheter. This arrangement limits obstructions to flow of blood through the catheter in the bypass segment, thereby maximizing flow and perfusion of the distal vascular territory during vessel obstruction by a blockage and during vessel obstruction by an inflated balloon or other obstruction. FIG. 6A is a transverse cross-sectional view of the catheter 80 showing one possible arrangement of the lumens, however, it should be appreciated that other arrangements of the lumens and sizes of the lumens can vary from those shown.

While the lumens disclosed in FIG. 6A as well as the lumens disclosed in each of the other embodiments described and illustrated in the drawings are each illustrated as including a circular (or generally circular) transverse cross-sectional configuration (e.g., diameter), alternative configurations are also contemplated herein. For example, it is envisioned that the lumens may each include a non-circular transverse cross-sectional configuration (e.g., square, rectangular, hexagonal, octagonal, pentagonal, a “house” silhouette, oval, elliptical, star-shaped, etc.). In the context of a star-shaped transverse cross-sectional configuration, any style of star may be used including, for example, a six-pointed star, a “Star of David,” etc. Such various lumen configurations are applicable to each of the catheters disclosed herein.

During use of the catheter 80, the distal region thereof is placed across the target tissue (e.g., a clot, a calcified heart valve, etc.) and blood is then able to bypass the target tissue (depending on the direction of blood flow) through the distal region. For example, it is envisioned that blood may flow through the side hole 88, through the distal region, and out of the distal end hole 86, or that blood may flow through the distal end hole 86, through the distal region, and out of the side hole 88. The catheter 80 may be configured such that the proximal end thereof is positioned externally of the patient (e.g., to allow for the aspiration of debris, clot material, etc., therethrough), whereby the proximal region of the catheter 80 does not service the bypass procedure.

In certain methods of use, if the side hole 88 is covered (e.g., by the patient's tissue), the bypass will be closed, and blood will not flow through the catheter 80. To establish blood flow, however, blood may be back bled or actively aspirated out of the body (e.g., via the proximal end of the catheter 80).

Similar to the secondary lumens of FIGS. 1 and 4, several alternate embodiments (not pictured) are also possible, including various arrangements that may incorporate the secondary and tertiary lumens within or substantially within the wall of the elongate member. Alternatively, a secondary lumen may be within or substantially within the wall, and a tertiary lumen may course through the primary central lumen, or vice-versa.

The ultrasonic source A provides ultrasonic energy through wire(s) 89 to the energy emitters 87 so that the medication flow within the blood clot is enhanced. Note three energy emitters (also referred to energy emitting elements) are shown by way of example as a fewer number or an additional number of emitters can be provided as well as spacing along a length of the catheter 80 other than the spacing shown can be utilized. Moreover, the emitters 87 are shown positioned on one side of the catheter 80, in a longitudinal row, but additional emitters can be provided on other sides of the catheter, e.g., a series or longitudinal row of emitters on sides of the catheter spaced 180 degrees apart from emitters 87 shown. It is also contemplated that rather than longitudinally spaced as shown, a series of emitters can be radially spaced along an outer wall of the catheter 80. Any combination of arrangements, on the catheter and/or in the catheter, as well as on a balloon or multiple balloons and/or in a balloon and/or multiple balloons are contemplated as well. In the embodiment of FIG. 6, the energy emitters 87 are adjacent the openings 83 which deliver the medication or therapeutic agent to the blood clot. In alternate embodiments, the energy emitters can be spaced from the openings 83.

In preferred embodiments, the emitters 87 are positioned between the side hole 88 and distal hole 86 as shown. However, it is also contemplated that one or more energy emitters 87 can in lieu of or in addition be placed proximal of the side hole 88. This can provide ultrasonic energy to regions of the vessel proximal of the blood clot.

The wires 89 transmit to the emitters 87 the ultrasonic energy from the ultrasonic transducer A which is remote from the emitters 87 as shown schematically in FIG. 6A. However, in alternate embodiments, the emitters can include ultrasonic transducers (which convert electrical energy into ultrasonic energy) which are connected via wires to an electrical energy source. The ultrasonic energy can be emitted as continuous waves and/or pulsed waves and in various shaped waveforms, e.g., sinusoidal. Various frequencies are also contemplated. A microcontroller can be provided to control output. Alternative forms of energy are also contemplated.

Temperature sensor(s) can be provided on or adjacent the emitters 87 to monitor temperature of the radiating elements 87 or tissue during the procedure. Cooling elements can be provided.

In use, the bypass catheter 80 is inserted, typically minimally invasively, and advanced through the vessels for placement adjacent the blood clot so that the side hole 88 is positioned proximal of the blood clot and the distal end hole 86 is positioned distal of the blood clot as in the bypass catheters discussed above. This enables blood flow from proximal of the clot past the clot to provide immediate and, if desired, continuous, blood flow (and tissue reperfusion) during the procedure, and for as long as the catheter is left in place. The medication source is opened for medication flow either by a valve or switch on the catheter 80 or at a remote location on or adjacent the medication source B. The radiating elements (emitters) 87 are also activated, either by a switch on the catheter 80 or a remote switch, e.g., at the energy source, to apply energy via wires 89 to emitters 87 to apply ultrasonic energy to the blood clot and/or vessel (generating an acoustic field) to increase the permeability in the blood clot to thereby increase the efficacy of the medication in dissolving the blood clot as the medication is driven deeper into the clot. The activation enhances mixing of the medication via pressure waves and/or cavitation. The ultrasound energy and fluid injection can in some embodiments be synchronized to occur simultaneously. Alternatively, energy and fluid injection can be applied at separate time/intervals. During the ultrasonic energy application and medication delivery, the side hole 88 remains open so blood flow can continue distal of the clot, thereby avoiding blood disruption which can cause ischemia or other adverse conditions.

Note that in alternate embodiments, the ultrasound energy can be used without the drug injection. In such embodiments, the pulsed sound waves created by the ultrasonic energy source and emitted by the radiating elements 87 fragments the blood clots via cavitation to mechanically break up the clot. In alternitive embodiments, rotational maceration can be used to mechanically break up the clot as well. As described previously, aspiration of clots and debris may optionally be performed as well. Combinations of the various techniques, either simultaneously and/or sequentially, may be performed as well.

In the alternate embodiment of FIG. 7, a pulse or shock wave generator C is connected to device (bypass catheter) 90. The generator produces shock waves that propagate through the blood clot to break up the clot. The pulse generator C can be utilized in some embodiments with medication to break up the clot. As noted above, alternatively the shock waves may be used to break up calcium or other hardenings.

More specifically, device 90 has one or more energy emitters 97 in the form of electrodes. As in device 80, device 90 has a distal exit (end) hole 96 and at least one side hole 98 in the outer wall of device 90. The bypass catheter 90 can be considered to have two segments or portions, either integral or separate joined components, as described above. The bypass catheter 90 in this embodiment has three channels (lumens) as in device 80: i) main channel 95a for fluid flow to bypass the blood clot; ii) channel 95b for injection of medication such as thrombolytics from fluid source B (via tubing 91) to dissolve the clot; and iii) channel 95c to contain the wire(s) 99 connecting the generator C to the electrodes 97 disposed on, e.g., along, the catheter 90. As described above with respect to lumens 85a, 85b and 85c, lumens 95a, 95b, 95c are provided by way of example and the variations described above for lumens 85a, 85b and 85c, and for the wires are fully applicable to lumens 95a, 95b and 95c of catheter 90 such as embedding in a wall of the catheter, centered, off-centered, etc.

During use of the catheter 90, the distal region thereof is placed across the target tissue (e.g., a clot, a calcified heart valve, etc.) and blood is then able to bypass the target tissue (depending on the direction of blood flow) through the distal region. For example, it is envisioned that blood may flow through the side hole 98, through the distal region, and out of the distal end hole 96, or that blood may flow through the distal end hole 96, through the distal region, and out of the side hole 98. The catheter 90 may be configured such that the proximal end thereof is positioned externally of the patient (e.g., to allow for the aspiration of debris, clot material, etc., therethrough), whereby the proximal region of the catheter 90 does not service the bypass procedure.

In certain methods of use, if the side hole 98 is covered (e.g., by the patient's tissue), the bypass will be closed, and blood will not flow through the catheter 90. To establish blood flow, however, blood may be back bled or actively aspirated out of the body (e.g., via the proximal end of the catheter 90).

Similar to the secondary lumens of FIGS. 1 and 4, several alternate embodiments are also contemplated, including various arrangements that may incorporate the secondary and tertiary lumens or electrodes or wires, or combinations thereof, within or substantially within the wall of the elongate member. Alternatively, a secondary lumen may be within or substantially within the wall of the catheter, and a tertiary lumen may course through the primary central lumen, or vice-versa.

The generator C provides voltage pulses (shock waves) transmitted by connectors or wire(s) 99 to the energy emitters (electrodes) 97 so that the shock waves propagate through the vessel and impinge on the blood clot to break up the clot. Note three energy emitters are shown by way of example as a fewer number or an additional number of emitters can be provided as well as spacing along a length of the catheter 90 other than the spacing shown can be utilized. Moreover, the emitters 97 are shown positioned on one side of the catheter 90, in a longitudinal row, but additional emitters can be provided on other sides of the catheter, e.g., a series or longitudinal row of emitters on sides of the catheter spaced 180 degrees apart from emitters 97 shown. It is also contemplated that rather than longitudinally spaced as shown, a series of emitters can be radially spaced along an outer wall of the catheter 90.

The generator C can be used in conjunction with medication flow through perforations 93 as in device 80, and the energy emitters 97 can be positioned adjacent the openings 83 which deliver the medication or therapeutic agent to the blood clot or alternatively spaced from the openings 83.

In preferred embodiments, the emitters 97 are positioned between the side hole 98 and distal hole 96 as shown. However, it is also contemplated that one or more energy emitters 97 can in lieu of or in addition be placed proximal of the side hole 98. This can provide shock waves to regions of the vessel proximal of the blood clot.

A microcontroller can be provided to control output. Temperature sensor(s) can be provided on or adjacent the emitters 97 to monitor temperature of the electrodes or tissue during the procedure. Cooling elements can be provided.

The bypass catheter 90 can be inserted and used in the same manner as catheter 80 except for the transmission of shock waves so the description of use of device 80 is applicable to the use of device 90. During the energy application (and medication delivery if provided) the side hole 98 remains open so blood flow can continue distal of the clot, thereby avoiding blood flow disruption which can cause ischemia or other adverse conditions.

Note that in alternate embodiments, the energy can be used without the drug injection. In such embodiments, the shock waves created by the energy source and emitted by the electrodes 97 fragments the blood clots via cavitation to mechanically break up the clot.

In some embodiments, a balloon can overlie the energy emitters and the pulses can be provided within the balloon. In some embodiments, the energy emitters in addition to or instead of being within the balloon can overlie a balloon and the pulses can be provided within or on the balloon. These external emitters are shown in the embodiment of FIG. 11 wherein energy emitters 124, e.g., electrodes, overlie balloon 125 which is attached to catheter 120. Catheter 120 includes a side hole 122 and a distal end hole 126 for blood bypass as in the other bypass catheters disclosed herein. The catheter also has a primary lumen for blood flow and a secondary lumen for inflation of balloon 125. The balloon 125 and energy emitters 124 are positioned between the side hole 122 and end hole 126 and the emitters emit energy to the vessel. Energy source emitter E is connected to the emitters 124 via wires 127 in the same manner as the other energy emitters disclosed herein. The energy emitters can be in the various forms disclosed herein.

During use of the catheter 120, the distal region thereof is placed across the target tissue (e.g., a clot, a calcified heart valve, etc.) and blood is then able to bypass the target tissue (depending on the direction of blood flow) through the distal region. For example, it is envisioned that blood may flow through the side hole 122, through the distal region, and out of the distal end hole 126, or that blood may flow through the distal end hole 126, through the distal region, and out of the side hole 122. The catheter 120 may be configured such that the proximal end thereof is positioned externally of the patient (e.g., to allow for the aspiration of debris, clot material, etc., therethrough), whereby the proximal region of the catheter 120 does not service the bypass procedure.

In certain methods of use, if the side hole 122 is covered (e.g., by the patient's tissue), the bypass will be closed, and blood will not flow through the catheter 120. To establish blood flow, however, blood may be back bled or actively aspirated out of the body (e.g., via the proximal end of the catheter 120).

The catheter 120 in the illustrated embodiment has a filter 130, however, it should be appreciated that catheter 120 can be provided without a filter. The filter 130 is attached to a wire 128 extending the length of the catheter for access to the clinician outside the patient at region 128a. Wires 133 support the filter material 131, and the filter terminates at region 132. The filter 130 can alternatively be attached or tethered to the catheter 120 as in filters 101 and 111 of FIGS. 9 and 10, respectively.

A sheath such as sheath 60 of FIG. 5 (or inner blocking member) can be provided to selectively open and close the side hole 88, 98, 122 of catheters 80, 90, 120 respectively (or the side holes of any of the other catheters disclosed herein), if it is desired to discontinue blood flow past the clot. In some embodiments, the catheter 80, 90, 120 can be connected to an aspiration source, such as aspiration source 39 described above in conjunction with the catheter of FIG. 5, to provide aspiration to effect backflow of blood through the catheter 80, 90 if and when desired.

Catheter 80, 90, 120 (as well as the other catheters disclosed herein) can include structure such as described above, e.g., a valve, restricted opening, etc., to restrict back flow through the catheter. Catheter 80, 90, 120 can also include anchoring structure such as wires or an inflatable balloon as in alternate embodiments described above.

Various forms of energy can be provided to the bypass catheter described herein such as ultrasonic energy, electrosurgical energy in the form of radiofrequency or microwave energy, etc. Furthermore, other types of energy including light or laser energy can be applied.

The energy can be applied between the side hole and distal hole so the clot can be treated while blood bypasses the clot as described above for immediate tissue reperfusion. That is, the various forms of energy and associated energy emitters or openings for energy emission in preferred embodiments are positioned between the side hole and distal exit hole. However, in alternate embodiments, instead of, or in lieu of energy applied between the side hole and distal hole, the energy emitters or openings can be positioned proximal of the side hole and/or have structure, e.g., an antenna or other energy emitting device, extending distal of the distal hole. When used with medication, the immediate reperfusion is beneficial as the clot lyses from lytic infusions over time.

In alternate embodiments, a mechanical thrombectomy device having at least one wire or other macerating structure is rotated by a motor positioned within the catheter such as in the embodiment of FIG. 8, or alternatively powered by a motor outside the catheter. The macerating element is mounted on a rotating shaft which upon actuation of the motor rotates about its axis so the macerating element breaks up the clot. The macerating element in preferred embodiments is positioned between the proximal and distal holes, however, it can alternatively be mounted in other locations. Alternatively, the macerating wires may be mounted on the catheter, and the entire catheter can be rotated for maceration.

In some embodiments, the catheter can have a complex shape to the second catheter segment, or a portion thereof, wherein rotation of the catheter itself can cause maceration. One example of such a complex shape is a sinusoidal shape.

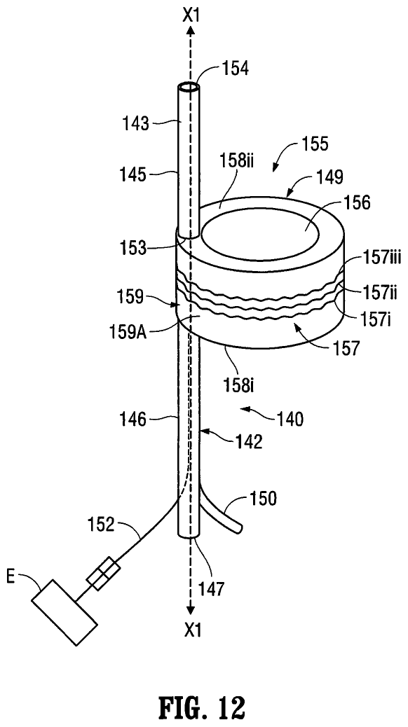

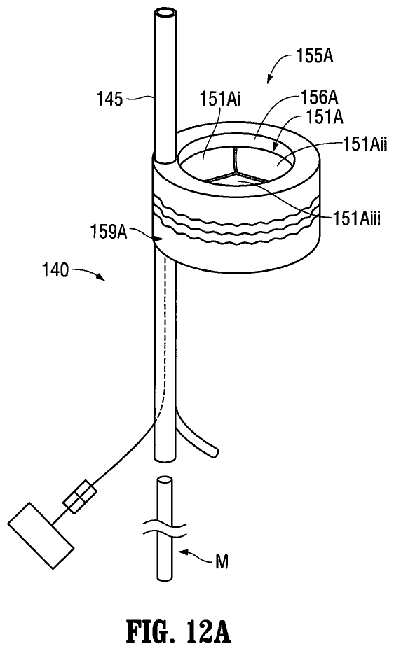

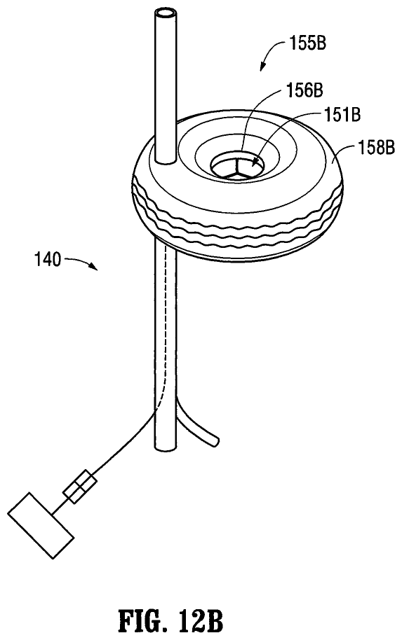

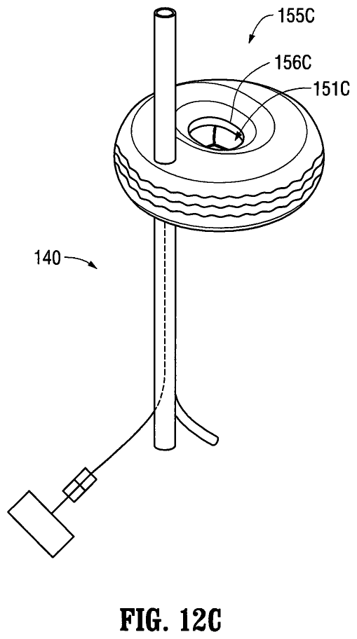

The bypass catheters disclosed hereinabove have a side hole(s) and a distal hole(s) for blood to bypass the blood clot or other vessel blockage. In the alternate embodiment of FIG. 12, a torus balloon is provided with a passageway for blood flow when the balloon is inflated. One type of balloon which can be utilized is the bulging torus balloon disclosed in U.S. Pat. No. 10,328,246, the entire contents of which are hereby incorporated herein by reference. Other shape balloons are also contemplated.

An energy source such as those described herein can provide energy to emitters, e.g., electrodes, positioned on or in the bulging torus balloon. This can be used during valve lithotripsy while allowing egress of blood from the heart through the central hole of the balloon, during prolonged balloon inflation for prolonged contact with the valve, or similarly continued blood flow through a vessel during use in a vessel, without critically obstructing blood flow.

FIG. 12 illustrates catheter 140 including a catheter body 142 having a (main, working) lumen 143 extending therethrough that is configured to receive one or more supplemental medical devices (e.g., catheters, instruments, tools, etc.) M and an energy delivery member 155 supported by (e.g., mounted on or otherwise secured to) the catheter body 142 such that the energy delivery member 155 extends radially outward therefrom. In the illustrated embodiment, the energy delivery member 155 is configured as an expandable member (e.g., a balloon) 149 and includes a body 159 with a cylindrical (or generally cylindrical) configuration that defines opposing (e.g., proximal and distal) end faces 158i, 158ii. In the illustrated embodiment, the end faces 158i, 158ii each include a planar (or generally planar) configuration, but non planar surfaces are also contemplated. It should be appreciated that alternate configurations for the energy delivery member 155 are also contemplated herein, as described in further detail below.

The energy delivery member 155 defines a channel 153 that is configured to receive the catheter body 142 such that the catheter body 142 extends therethrough as illustrated. The region of the catheter 140 distal of the energy delivery member 155 is designated by reference numeral 145 and the region of catheter 140 proximal of the energy delivery member 155 is designated by reference numeral 146. It should be appreciated that the energy delivery member 155 may be located at any suitable location along the catheter body 142. The catheter has a proximal opening 147 and a distal opening 154 fluidly connected by a lumen 143 e.g., primary central lumen. Port 150 provides for injection of fluid (liquid or gas) to expand (e.g., inflate) the energy delivery member 155.

The energy delivery member 155 includes a passageway 156 (e.g., an opening, a channel, a conduit, etc.) that extends through the body 159 in parallel (or generally parallel) relation to a longitudinal axis X1 defined by the catheter body 142 to allow blood to flow through the energy delivery member 155 (e.g., subsequent to expansion) during the course of a surgical procedure. Although illustrated as being centrally positioned in the particular embodiment seen in FIG. 12, it is also envisioned that the passageway 156 may be eccentrically positioned (e.g., such that the passageway 156 is radially offset from a center of the energy delivery member 155), as described in further detail below.

The energy delivery member 155 is eccentrically mounted, whereby the energy delivery member 155 and the passageway 156 are each radially offset from (out of alignment with) the longitudinal axis X1 defined by the catheter body 142 (e.g., such that a majority (more than 50%) of the energy delivery member 155 and the passageway 156 are offset to one side of the longitudinal axis X1).

One or more energy emitters 157 are supported by the energy delivery member 155 adjacent to an outer surface (wall, circumference) 159A of the body 159 and are configured to communicate energy to target tissue to facilitate treatment thereof. In the particular embodiment illustrated, for example, the energy delivery member 155 includes a plurality of energy emitters 157i, 157ii, 157iii that extend along the circumference thereof. Although illustrated as including three energy emitters 157 in the particular embodiment seen in FIG. 12, it should be appreciated that the number of energy emitters 157 may be increased or decreased without departing from the scope of the present disclosure. For example, an embodiment of the energy delivery member 155 including a single energy emitter 157 is also contemplated herein.

Expansion (e.g., inflation) of the energy delivery member 155 brings the energy emitter(s) 157 closer to the vessel blockage for emission of energy to treat the blood clot or other vessel blockage. The energy emitter(s) 157 may be arranged in a variety of arrays and spaced in any suitable manner (e.g., along the circumference of the energy delivery member 155). The energy emitter(s) 157 can emit ultrasonic energy or other energy, and at various frequencies, as in the other energy emitters disclosed above. Connector 152 (e.g., one or more wires) connects the energy emitter(s) 157 to an external energy source E, which allows energy to be applied to the energy delivery member 155 by communicating energy from the external energy source E to the energy emitter(s) 157. In the illustrated embodiment, the connector 152 extends internally within (through) the catheter body 142 (e.g., via the lumen 143). It is also envisioned, however, that the connector 152 may extend within or substantially within a wall of the catheter or extend externally of the catheter body 142 in various embodiments of the disclosure.

The energy emitter(s) 157 can be positioned on the outer surface 159A of the body 159 of the energy delivery member 155, as illustrated in FIG. 12. Alternatively, it is envisioned that the energy emitter(s) 157 may extend onto the outer surface 159A from within the energy delivery member 155 or that the energy emitter(s) 157 may be located internally (within) the energy delivery member 155. For example, the energy emitter(s) 157 may be embedded within the material used in construction of the energy delivery member 155 such that they are positioned radially inward of the outer surface 159A. In such embodiments, the energy emitter(s) 157 may be exposed via one or more openings (e.g., windows) formed in the outer surface 159A of the body 159.

In use, the energy delivery member 155 is introduced across target tissue (e.g., a valve or other such targeted site), and the energy delivery member 155 is expanded (e.g., inflated). When expanded (e.g., to fill a vessel lumen), blood flow is maintained through the passageway 156 extending through the energy delivery member 155. Energy is then applied to the energy emitter(s) 157 from the energy source E over a period of time to treat the target tissue. Following treatment, the transmission of energy to the energy emitter(s) 157 is ceased and the energy delivery member 155 is collapsed (e.g., deflated), which allows for removal of the catheter 140. In some embodiments, these steps of balloon inflation and energy delivery may be repeated two or more times before removal of the catheter.

In some embodiments, the energy delivery member 155 is expanded (e.g., inflated) such that the energy emitter(s) 157 are in contact with the target tissue (e.g., a blockage or calcifications in the lumen of a vessel). In other embodiments, the energy delivery member 155 may be configured such that the energy emitter(s) 157 are spaced from (out of contact with) the target tissue subsequent to expansion (e.g., inflation).

In some embodiments, the catheter body 142 may define a single lumen 141, as seen in FIG. 12, to allow for the passage of a wire therethrough, the injection of fluid to the target site, expansion and collapse (e.g., inflation and deflation) of the energy delivery member 155, etc. In other embodiments, the catheter body 142 may include a separate, dedicated lumen that is configured to support expansion and collapse (e.g., inflation and deflation) of the energy delivery member 155, which may extend through (within) an outer wall of the catheter body 142. It is also envisioned that the catheter body 142 may include multiple energy delivery members 155, as discussed in further detail below, each of which may be fed by a separate lumen.

Catheter 140 can have a filter at a distal end as in the catheters described above.

The energy delivery member 155 is capable of prolonged expansion (e.g., inflation) within a cardiac valve, a vessel, or other regions without critically obstructing outflow/blood flow due to the presence of the passageway 156. Without the passageway 156, blood flow would be obstructed (or blocked entirely), which has adverse consequences if cut off for a long period of time, especially in surgical procedures such as cardiac valve procedures. The bypass catheters disclosed herein (e.g., the bypass catheters having balloon mounted or carried energy emitters), are likewise capable of prolonged expansion (e.g., inflation) within the valve such as a heart valve, e.g., cardiac valve, a vessel, or other regions without critically obstructing outflow/blood flow as the blood flows into the side hole and exits the distal hole. It is envisioned that the catheters disclosed herein may be used for intravascular or intraluminal lithotripsy to break down calcium (e.g., via the aforedescribed energy emitter(s) 157 mounted or carried by the energy delivery member 155) while providing a channel/passage for blood flow (e.g., via the passageway 156). For example, the catheters described herein may be used pre-operationally to soften any such calcium deposits.

While blood is allowed to flow through the energy delivery member 155 during the course of the procedure via the passageway 156, blood flow may still be limited (e.g., due to backflow (retrograde blood flow) form the aorta into the left ventricle). To address this concern, in an alternate embodiment, the catheter 140 may include an energy delivery member 155A (FIG. 12A) with a body 159A defining a passageway 156A that includes (supports) a valve 151A so as to inhibit (if not entirely prevent) retrograde blood flow (e.g., backflow) through the energy delivery member 155A (e.g., to thereby extend the time available for completion of a lithotripsy or other such surgical procedure). In the illustrated embodiment, the valve 151A includes a tricuspid configuration with leaflets 151Ai, 151Aii, 151Aiii. It should be appreciated, however, that the configuration of the valve 151A may be varied in alternate embodiments without departing from the scope of the present disclosure, as elaborated upon below.

Valve 151A, like other valves disclosed herein can open and close, and in some embodiments open intermittently. The open valve allows blood flow through the valve. In some embodiments, a pressure gradient across the valve causes the valve to open.

The valves disclosed herein can be used to inhibit blood flow during diastole so longer treatments can be used without symptomatic heart flow. (Since blood flow cannot be stopped during systole or patient will die).

FIG. 12B illustrates another embodiment of the disclosure in which the catheter 140 includes an energy delivery member 155B with a toroidal (or generally toroidal, torus configuration) that defines an outer surface 158B with a continuous curvature. While the energy delivery member 155B is illustrated as including a passageway 156B and a valve 151B with a circular (or generally circular) transverse cross-sectional configuration (e.g., diameter), it should be appreciated that the particular configurations of the passageway 156B and the valve 151B may be varied in alternate embodiments without departing from the scope of the present disclosure. For example, FIG. 12C illustrates an alternate embodiment of the energy delivery member 155B, which is identified by the reference character 155C, that includes a passageway 156C and a valve 151C with noncircular (e.g., elliptical) transverse cross-sectional configurations.

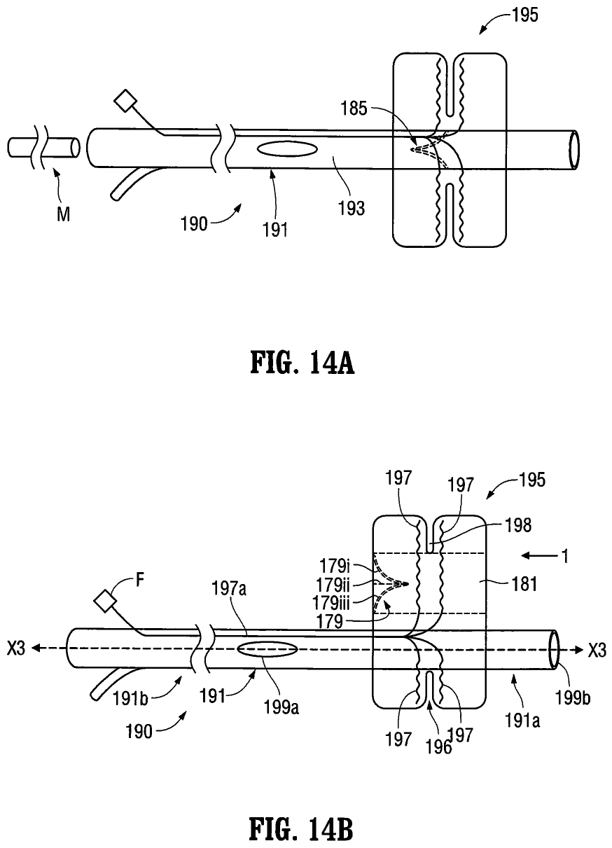

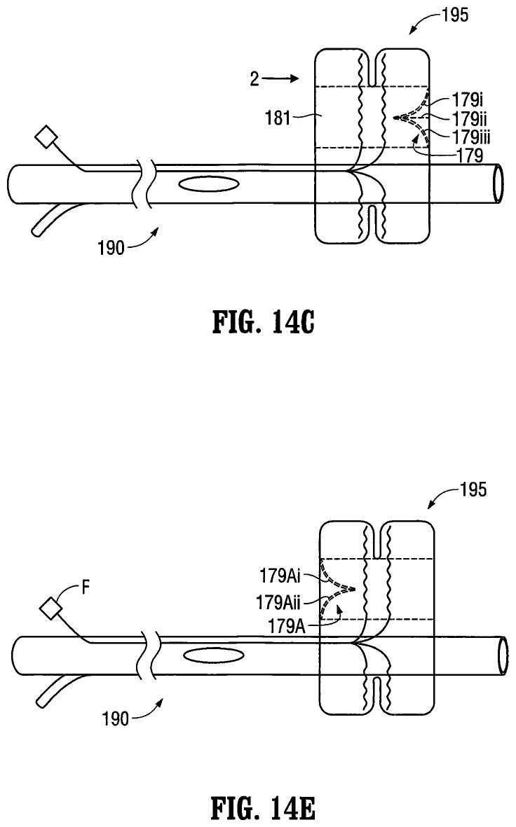

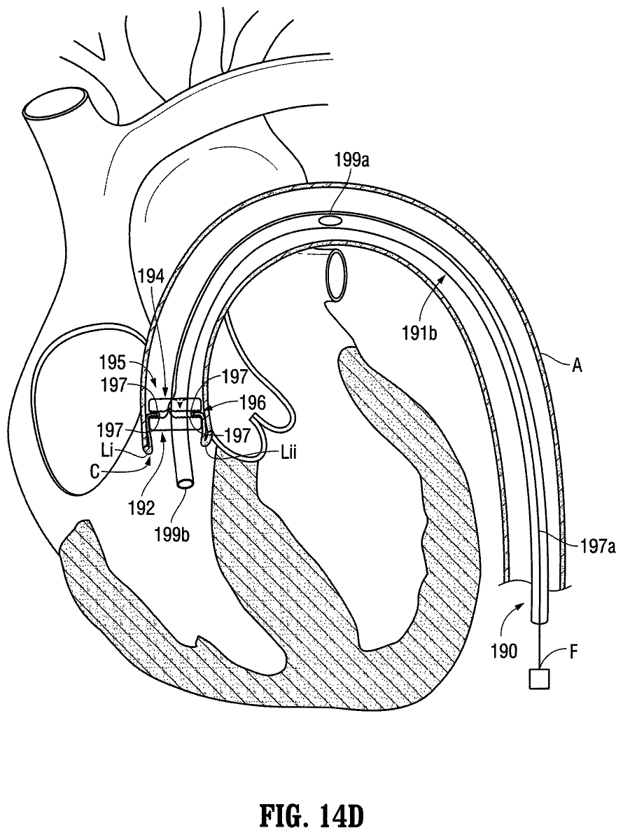

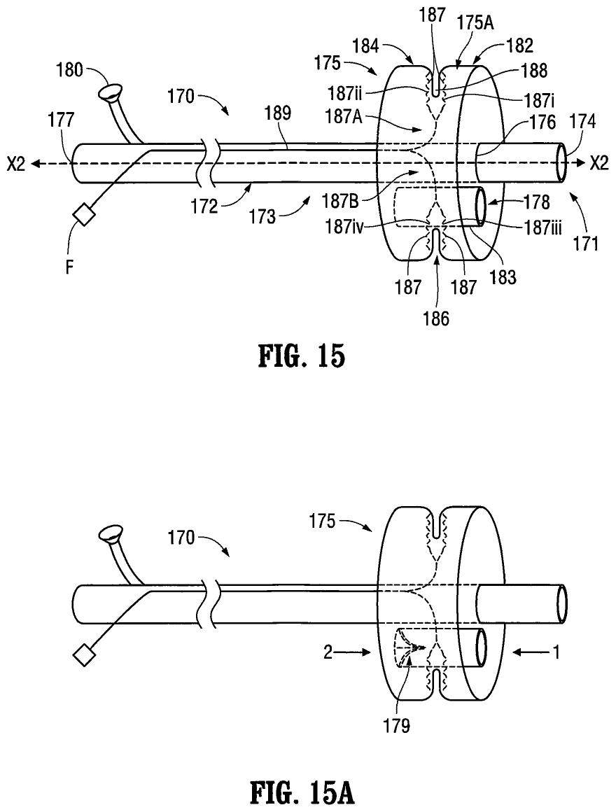

Conceptually when a valve is stenotic, the outer portions of the leaflets fuse, and the remaining hole is narrowed. When a TAVR (endovascular valve replacement) is performed, this narrowed valve is stretched open, either with a balloon or a self-expanding valve. But if the fused section of the valve is highly calcified, the valve and adjacent tissue and/or aortic root can crack when it is stretched, which is usually fatal. The catheters of the present invention can be utilized to soften the calcium with lithotripsy before the TAVR so it can protect against this catastrophic complication. If the balloon (or other such expandable member) is only in the small hole, the contact area for the lithotripsy is very limited. In contrast, if the surface area can be increased, calcifications can be reduced, the catheters of FIGS. 14-15 achieve this by providing an increased surface area for lithotripsy. These catheters have energy emitters mounted on balloon portions which are placed on opposing sides of the valve.

More specifically, FIG. 15 illustrates a catheter 170 including a catheter body 172 and an energy delivery member 175 mounted on (or otherwise secured to) the catheter body 172 such that the energy delivery member 175 extends radially outward therefrom. In the illustrated embodiment, the energy delivery member 175 is configured as an expandable member (e.g., a balloon) 175A with a (generally) cylindrical configuration that defines a channel 176 that is configured to receive the catheter body 172 such that the catheter body 142 extends therethrough as illustrated. The region of the catheter 170 distal of the energy delivery member 175 is designated by reference numeral 171 and the region of catheter 170 proximal of the energy delivery member 175 is designated by reference numeral 173. It should be appreciated that the energy delivery member 175 may be located at any suitable location along the catheter body 172. The catheter 170 has a proximal opening 177 and a distal opening 174 fluidly connected by a lumen extending within the catheter 170. Port 180 provides for injection of fluid (liquid or gas) to expand (e.g., inflate) the energy delivery member 175.

The energy delivery member 175 includes a passageway 178 (e.g., an opening, a channel, a conduit, etc.) that extends in parallel (or generally parallel) relation to a longitudinal axis X2 defined by the catheter body 172 to allow blood to flow through the energy delivery member 175 (e.g., subsequent to expansion) during the course of a surgical procedure. In the particular embodiment illustrated, the passageway 178 is defined by a tubular member (portion, sleeve) 183 with proximal and distal openings that extends through the energy delivery member 175. The tubular member 183 and, thus, the passageway 178, is eccentrically positioned and is radially offset from the channel 176 and a center of the energy delivery member 175, whereby a majority (e.g., more than 50%) of the tubular member 183 is offset to one side of the longitudinal axis X2 defined by the catheter body 172.

In certain embodiments, the catheter 170 may include a valve 179 that is located within the passageway 178 (e.g., within the tubular member 183), as seen in FIG. 15A. The valve 179 is similar (if not identical to) the valve 151 discussed above with respect to FIG. 12A and is configured to inhibit (if not entirely prevent) blood flow through the energy delivery member 175. For example, the valve 179 may be configured to inhibit (if not entirely prevent) retrograde blood flow (e.g., backflow) through the energy delivery member 175 (e.g., in the proximal direction indicated by the arrow 1), as seen in FIG. 15A. It is also envisioned, however, that the configuration of the valve 179 may be reversed (inverted) such that the valve 179 is configured to inhibit (if not entirely prevent) antegrade blood flow (e.g., in the distal direction indicated by the arrow 2) through the energy delivery member 175.

Upon expansion (e.g., inflation), the energy delivery member 175 defines (includes): a first (distal) portion 182; a second (proximal) portion 184; and an intermediate portion 186 that is located between the respective first and second portions 182, 184. The intermediate portion 186 forms a waist or narrowed portion with a gap 188 that is configured to receive target tissue (e.g., a patient's valve) upon expansion (e.g., inflation) of the energy delivery member 175. That is, a transverse dimension of the intermediate (narrowed) portion 186 is less than a transverse dimension of the first portion 182 and the second portion 184. In this manner, the respective first and second portions 182, 184 can be placed on opposing sides of the target tissue with at least one energy emitting surface configured to press against opposite sides of the target tissue (e.g., to treat a valve such as a cardiac valve) and configured for placement on different sides of the waist as described below.

One or more energy emitters 187 are supported by the energy delivery member 175 adjacent to an outer surface thereof, which are configured to communicate energy to target tissue to facilitate treatment thereof, as discussed above in connection with the catheter 140 seen in FIG. 12. In the particular embodiment illustrated, for example, the energy delivery member 175 includes a plurality of energy emitters 187 that extend along the circumference thereof. More specifically, energy delivery member 175 includes energy emitters 187 that are supported (located, positioned) on the second portion 182 so as to face the first portion 184 and energy emitters 187 that are supported (located, positioned) on the first portion 184 so as to face the second portion 182. It is envisioned that one or more energy emitters 187 may be provided on each of the respective first and second portions 182, 184 (in any suitable location).

Expansion (e.g., inflation) of the energy delivery member 175 brings the energy emitters 187 closer to the target tissue to facilitate the delivery of energy thereto during treatment (e.g., to reduce calcium). The energy emitters 177 can emit ultrasonic energy or other energy, and at various frequencies, as in the other energy emitters disclosed above.

Connector 189 (e.g., one or more wires) connects the energy emitters 187 to an external energy source F. The energy emitters 187 can be positioned on the outer wall (circumference) of the energy delivery member 175. Alternatively, the energy emitters 187 may extend onto the outer wall from within the energy delivery member 175, or the energy emitters 187 may be located internally (within) the energy delivery member 175. For example, the energy emitters 187 may be embedded within the material used in construction of the energy delivery member 175. In such embodiments, the energy emitters 187 may be exposed via one or more openings (e.g., windows) defined by the energy delivery member 175.

It is envisioned that the energy emitters 187 may be arranged in a variety of arrays and spaced in a variety of manners in various embodiments of the disclosure to achieve any necessary or desired therapeutic effect (e.g., depending upon the particular nature and/or the particular location of the abnormality being treated). In the particular embodiment of FIG. 15, for example, the energy emitters 187 extend from the connector 189 so as to define a (bifurcated) first branch 187A with (first and second) legs 187i, 187ii and a (bifurcated) second branch 187B with (third and fourth) legs 187iii, 187iv, wherein the legs 187i, 187iii are arranged on the first portion 182 of the energy delivery member 175 and the legs 187ii, 187iv are arranged on the second portion 184 of the energy delivery member 175. As seen in FIG. 15, the legs 187i, 187ii extend in a first (circumferential) direction and the legs 187iii, 187iv extend in a second, opposite (circumferential) direction. In various embodiments of the disclosure, the energy emitters 187 may extend from the connector 189 so as to partially or entirely circumscribe the energy delivery member 175.

In some embodiments, the energy delivery member 175 is expanded (e.g., inflated) such that the energy emitters 187 are in contact with the target tissue (e.g., the blockage or calcifications in the lumen of the vessel). In other embodiments, the energy delivery member 175 may be configured such that the energy emitter 187 are spaced from (out of contact with) the target tissue subsequent to expansion (e.g., inflation).

In use, the energy delivery member 175 is introduced across target tissue (e.g., a valve or other such targeted site), and the energy delivery member 175 is expanded (e.g., inflated). Upon expansion, the energy delivery member 175 is positioned such that second portion 182 is located adjacent to (e.g., in contact with) a first (distal) side of the target tissue and the first portion 184 is located adjacent to (e.g., in contact with) a second (e.g., proximal) side of the target tissue such that the energy delivery member 175 spans (straddles) the target tissue, whereby the waist formed by the intermediate portion 186 receives the target tissue (e.g., such that the valve orifice and/or the valve leaflets are located within the gap 188). In some embodiments, the energy delivery member 175 may be configured such that the respective first and second portions 182, 184 may be spaced (e.g., axially) from the target tissue (e.g., sides of the valve and/or the valve leaflets). Alternatively, the energy delivery member 175 may be configured such that the respective first and second portions 182, 184 may be in contact with the target tissue (e.g., sides of the valve and/or the valve leaflets). For example, the energy delivery member 175 may be configured such that the respective first and second portions 182, 184 form kissing sides that press against opposite sides of the valve (e.g., the valve leaflets).

During use of the catheter 170, following expansion (e.g., inflation) of the energy delivery member 175, energy is applied to the target tissue via the energy emitters 187 over a period of time. The configuration of the energy delivery member 175 and the orientation of the energy emitters 187 increases the surface area of target tissue contacted by the energy delivery member 175. For example, in the context of a calcified valve, the increased surface area contacted by the energy delivery member 175 significantly decreases the load of brittle calcium in the valve and the surrounding tissue. When expanded (e.g., inflated), although the energy delivery member 175 may fill the vessel lumen, blood flow is maintained through the passageway 178 in the energy delivery member 175.

Following treatment, the transmission of energy to the energy emitter(s) 187 is ceased and the energy delivery member 175 is collapsed (e.g., deflated), which allows for removal of the catheter 170. In some embodiments, these steps of emitting energy and inflating may be repeated two or more times.

With reference now to FIG. 14, another embodiment of the catheter will be discussed, which is identified by the reference character 190. The catheter 190 includes a catheter body 191 having an energy delivery member 195 and a (main, working) lumen 193 extending therethrough that is configured to receive one or more supplemental medical devices (e.g., catheters, instruments, tools, etc.).

The energy delivery member 195 is supported by (e.g., mounted on or otherwise secured to) the catheter body 191 such that the energy delivery member 195 extends radially outward therefrom and is substantially similar to the aforedescribed energy delivery member 175 (FIG. 15). More specifically, upon expansion (e.g. inflation), the energy delivery member 195 defines a cylindrical (or generally cylindrical) configuration and includes: a first (distal) portion 192 defining a (distal) end face 192A that is planar (or generally planar) in configuration; a second (proximal) portion 194 defining a (proximal) end face 194A that is planar (or generally planar) in configuration; and an intermediate portion 196 that is located between the respective first and second portions 192, 194. It should be appreciated, however, that alternate configurations for the energy delivery member 195 are also contemplated herein, as described in further detail below.

The intermediate portion 196 forms a waist or narrowed portion with a gap 198 that is configured to receive target tissue (e.g., a patient's valve). That is, a transverse dimension of the intermediate (narrowed) portion is less than a transverse dimension of the first portion 192 and the second portion 194. In this manner, the respective first and second portions 192, 194 can be placed on opposing sides of the target tissue (e.g., the valve leaflets) in the same manner as energy delivery member 175.

In the particular embodiment illustrated in FIG. 14, the energy delivery member 195 is centrally positioned (concentrically mounted) relative to a longitudinal axis X3 defined by a body 193 of the catheter 190. It is also envisioned, however, that the energy delivery member 195 may be eccentrically positioned (e.g., such that a center of the energy delivery member 195 is radially offset from the longitudinal axis X3). In contrast to the catheter 170, the catheter 190 is configured and functions as a bypass catheter (similar to that illustrated in FIG. 1) in that the energy delivery member 195 is devoid of a blood flow passageway (e.g., the passageway 178 discussed above). Instead, the catheter body 191 includes a side hole 199a a distal end hole 199b (which may be similar or identical to the aforementioned side hole 2 and end hole 4 (FIG. 1), respectively), which facilitates blood flow into the side hole 199a, through the catheter body 191, and out of the distal end hole 199b in the same manner as the bypass catheters described herein.

The energy delivery member 195 includes energy emitters 197 which are similar. or identical to the energy emitters 187 discussed above in connection with the catheter 170 (FIG.15) and are in communication with an external energy source F via a connector 197a. The energy emitters 197 are configured and positioned to direct energy into the gap 198 upon receipt of the target tissue to facilitate treatment thereof. More specifically, the energy delivery member 195 includes energy emitters 197i, 197ii, which are supported on (by) the second portion 194 so as to face the first portion 192, and energy emitters 197iii, 197iv, which are supported on (by) the first portion 192 so as to face the second portion 194, whereby the energy emitters 197i, 197ii and the energy emitters 197iii, 197iv face in opposite (or generally opposite) directions.

The catheter 190 and the energy delivery member 195 are used in a similar manner as the catheter 170 (FIG. 15) and the energy delivery member 175 regarding placement with respect to target tissue (e.g., a cardiac valve). In contrast to the catheter 170, however, during use of the catheter 190, blood flows through the side hole 199a to the distal hole 199b (via the catheter body 191), rather than through the energy delivery member 195 itself, which also reduces (if not entirely eliminates) critical flow obstruction upon expansion (e.g., inflation) of the energy delivery member 195. The energy emitters 197iii, 194iv (like the energy emitters of FIGS. 14A-16A) are on a proximally facing surface of distal portion 192, opposite distal facing surface 192A, and the energy emitters 197i, 197ii, (like the energy emitters of FIGS. 14-16A) are on a distally facing surface of proximal portion 194, opposite proximal facing surface 194A.

FIG. 14A illustrates an alternate embodiment of the (bypass) catheter 190, which includes a valve 185 that is located (positioned, supported) within the lumen 193 defined by the catheter body 191 (e.g., to sealingly receive the supplemental medical device M and/or inhibit blood flow through the catheter 190). In all other respects, the catheter of FIG. 14A is the same as the catheter of FIG. 14. Although illustrated as being located adjacent (or generally adjacent) to the energy delivery member 195 (e.g., such that the energy delivery member 195 spans the valve 185), it should be appreciated that the particular location of the valve 185 may be altered in various embodiments without departing from the scope of the present disclosure. For example, embodiments in which the valve 185 may be located proximally or distally of the energy delivery member 195 are also contemplated herein, as discussed in further detail below.

FIG. 14B illustrates another embodiment of the (bypass) catheter 190, in which the energy delivery member 195 includes a passageway 181 and the aforedescribed valve 179. To accommodate for inclusion of the passageway 181, the gap 198 defined by the intermediate portion 196 of the energy delivery member 195 is shortened and defines a reduced radial dimension (length) (when compared to the embodiments of the catheter 190 seen in FIGS. 14 and 14A, for example).

The valve 179 is located (positioned, supported) within the passageway 181 and is configured to inhibit (if not entirely prevent) blood flow through the energy delivery member 195 via the passageway 181. In the particular embodiment of the disclosure seen in FIG. 14B, the valve 179 is configured to inhibit (if not entirely prevent) retrograde blood flow (e.g., backflow) through the energy delivery member 195 (e.g., in the proximal direction indicated by the arrow 1). It is also envisioned, however, that the configuration of the valve 179 may be reversed (inverted), as seen in FIG. 14C, such that the valve 179 is configured to inhibit (if not entirely prevent) antegrade blood flow (e.g., in the distal direction indicated by the arrow 2) through the energy delivery member 195.

In the particular embodiment of the disclosure illustrated in FIG. 14B, the energy delivery member 195 is eccentrically mounted, whereby the energy delivery member 195 and the passageway 181 are each radially offset from (out of alignment with) the longitudinal axis X3 defined by the catheter body 191 (e.g., such that a majority (more than 50%) of the energy delivery member 195 and the passageway 181 are positioned eccentrically relative to the longitudinal axis X3 and are offset to one side thereof). Embodiments are also envisioned, however, in which the energy delivery member 195 may be concentrically positioned about the catheter body 191 while the passageway 181 remains radially offset from the longitudinal axis X3.

With reference to FIG. 14D, during use, the catheter 190 is advanced towards the target tissue (e.g., a patient's cardiac valve C) through an access vessel A (e.g., the patient's aorta) via a femoral or radial approach until the distal portion 192 of the energy delivery member 195 is positioned distally of the target tissue and the proximal portion 194 of the delivery member 195 is positioned proximally of the target tissue. When so positioned, the target tissue (e.g., the leaflets Li, Lii of the cardiac valve C) is received within the gap 198 (FIG. 14B) defined by the intermediate portion 196 of the energy delivery member 195 such that the energy emitters 197 are positioned adjacent (or generally adjacent) to the target tissue (e.g., such that the energy emitters 197 are in contact therewith). Energy is then communicated to the target tissue from the external energy source F via the connector 197a and the energy emitters 197 to thereby treat the target tissue (e.g., soften any calcium deposits on the valve leaflets Li, Lii).

When the catheter 190 is positioned in the manner illustrated in FIG. 14D, a distal region 191a thereof spans (extends across, distally beyond) the target tissue, which allows blood to bypass the target tissue (depending on the direction of blood flow) through the distal region 191a. For example, it is envisioned that blood may flow through the side hole 199a, through the distal region 191a, and out of the distal end hole 199b, or that blood may flow through the distal end hole 199b, through the distal region 191a, and out of the side hole 199a. The catheter 190 may be configured such that the proximal end thereof is positioned externally of the patient (e.g., to allow for the aspiration of debris, clot material, etc., therethrough), whereby a proximal region 191b of the catheter 190 does not service the bypass procedure. Note side hole 199a (and other side holes for blood flow in other catheters disclosed herein) can be positioned in portions of the catheter other than that shown in FIG. 14D.

In certain methods of use, if the side hole 199a is covered (e.g., by the access vessel A or other sections of the patient's tissue), the bypass will be closed, and blood will not flow through the catheter 190. To establish blood flow, however, blood may be back bled or actively aspirated out of the body (e.g., via the proximal end of the catheter 190).

FIG. 14E illustrates another embodiment of the (bypass) catheter 190, in which the energy delivery member 195 includes a valve 179A. In contrast to the valve 179 seen in FIGS. 14B and 14C, which includes a tricuspid configuration defining leaflets 179i, 179ii, 179iii that is similar (if not identical) to the valve 151 discussed above with respect to FIG. 12A, the valve 179A include a bicuspid configuration defining leaflets 179Ai, 179Aii.

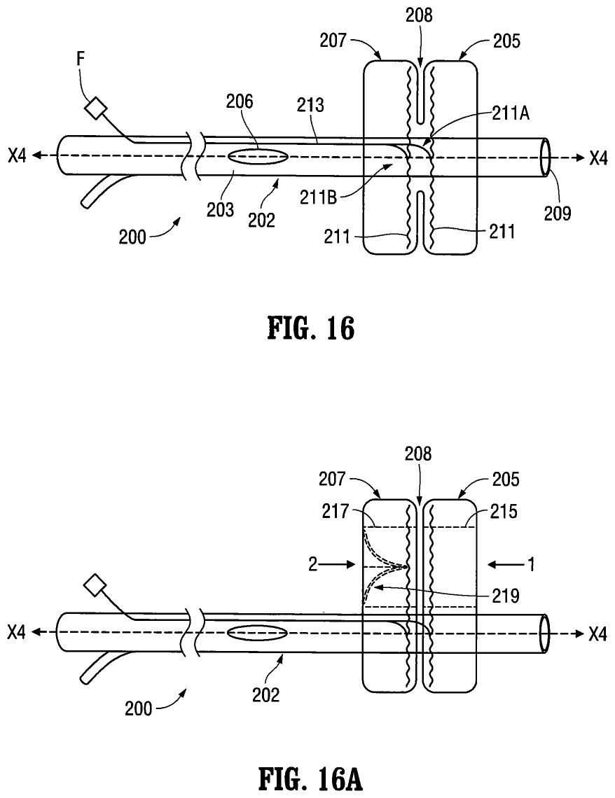

FIG. 16 illustrates another embodiment of the presently disclosed (bypass) catheter, which is identified by the reference character 200. The catheter 200 includes: a catheter body 202 defining a longitudinal axis X4 and a (main, working) lumen 203 that is configured to receive one or more supplemental medical devices (e.g., catheters, instruments, tools, etc.) M (FIG. 14A); a distal energy delivery member 205; and a proximal energy delivery member 207. The energy delivery members 205, 207 are arranged in a conjoined configuration so as to define a space (gap) 208 therebetween and are each centrally positioned about (concentrically mounted to) the catheter body 202.

During use of the catheter 200, the distal energy delivery member 205 is placed on one side of the target tissue (e.g., the valve leaflets) and the proximal energy delivery member 207 is placed on the other side of the target tissue. The space (gap) 208 is formed by the axial spacing of the energy delivery members 205, 207, rather than by a narrowed portion, as discussed above in connection with FIG. 15, for example. The energy delivery members 205, 207 each include one or more energy emitters 211 that are connected to the external energy source F by a connector 213. The energy emitters 211 are arranged in opposing relation such that the energy emitter(s) 211 included on the energy delivery member 205 are oriented towards (face) the energy delivery member 207 and such that the energy emitter(s) 211 included on the energy delivery member 207 are oriented towards (face) the energy delivery member 205. The catheter 200 functions in the same manner as the aforedescribed catheter 190 with respect to placement of the energy delivery members 205, 207 (one on each side of the valve) and blood flow through the catheter 200. More specifically, blood flows through a side hole 206, through the lumen 203 defined by the catheter body 202, and through a distal end hole 209.

As discussed above with respect to the catheter 170 (FIG. 15), it is envisioned that the energy emitters 211 may be arranged in a variety of arrays and spaced in a variety of manners to achieve any necessary or desired therapeutic effect (e.g., depending upon the particular nature and/or the particular location of the abnormality being treated). In the particular embodiment of the disclosure seen in FIG. 16, for example, the energy emitters 211 extend from the connector 213 so as to define a (continuous, non-bifurcated) first (distal) branch 211A that extends circumferentially about the energy delivery member 205 (either partially or entirely) and a (continuous, non-bifurcated) second (proximal) branch 211B that extends circumferentially about the energy delivery member 207 (either partially or entirely).

Note that shapes other than those discussed in connection with the aforedescribed energy delivery members could be utilized to treat the target tissue (e.g., valves), as mentioned above.

It is envisioned that the energy delivery members 175, 195, 205 etc. can alternatively have a figure eight configuration.

Note the catheter of FIGS. 15-16 are described for use with valves such as cardiac valve, but can be used to treat other area/tissue of a patient, while maintaining blood flow.

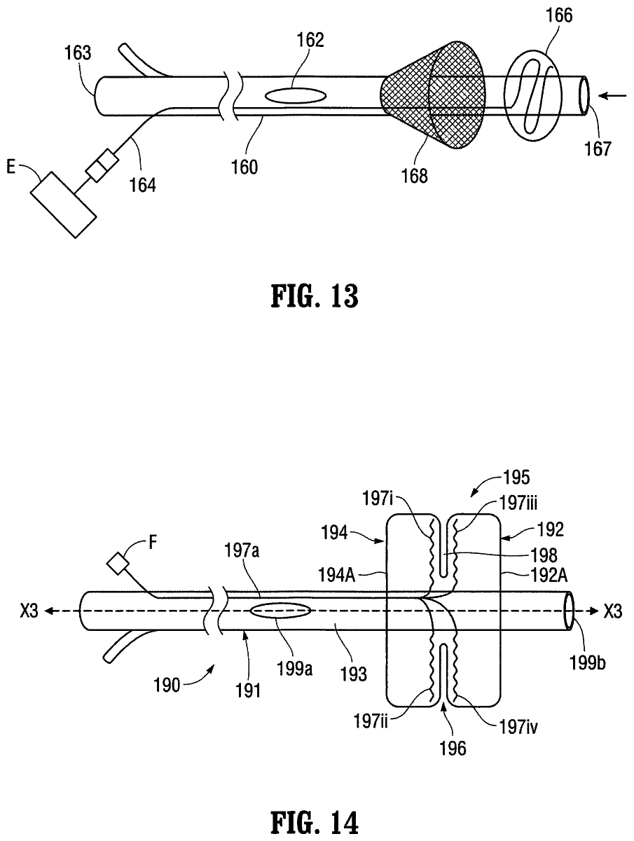

In some embodiments of the devices disclosed herein, when the device is introduced from a retrograde ‘upstream” approach blood may flow through the device in the opposite direction. This is depicted for example in FIG. 13 wherein blood flows through distal opening 167 of catheter in the direction of the arrow and can exit side hole 162 and/or proximal hole 163. The catheter 160 can include an energy delivery member 166 (e.g., a balloon or other such expandable structure) with energy emitters connected via wire 164 to an external energy source E as in the aforedescribed embodiments. Catheter 160 can also include a filter 168 as in the embodiments described above proximal or distal of the energy emitters.

FIG. 16A illustrates another embodiment of the (bypass) catheter 200 in which the distal energy delivery member 205 and the proximal energy delivery member 207 are configured as separate (discrete) structures that are spaced axially from each other along the longitudinal axis X4 defined by the catheter body 202 so as to define the space (gap) 208. The energy delivery members 205, 207 respectively define (first and second) passageways 215, 217, which are similar (if not identical to) the aforedescribed passageways 156 (FIGS. 12, 12A), 181 (FIG. 14B).

In the particular embodiment of FIG. 16A, the energy delivery members 205, 207 are eccentrically mounted, whereby the energy delivery members 205, 207 and the passageways 215, 217 are each radially offset from (out of alignment with) the longitudinal axis X4 defined by the catheter body 202 (e.g., such that a majority (more than 50%) of the energy delivery members 205, 207 and the passageways 215, 217 are offset to one side of the longitudinal axis X4). Embodiments are also envisioned, however, in which the energy delivery members 205, 207 may be concentrically positioned about the catheter body 202 while the passageways 215, 217 remains radially offset from the longitudinal axis X3.

As seen in FIG. 16A, the energy delivery members 205, 207 are configured such that the passageways 215, 217 are (generally) oriented in radial alignment, which facilitates blood flow through the energy delivery members 205, 207. More specifically, in the particular embodiment illustrated, the energy delivery members 205, 207 are configured to allow for antegrade blood flow therethrough (e.g., in the distal direction indicated by arrow 2) while retrograde blood flow (e.g., backflow in the proximal direction indicated by the arrow 1) is inhibited (if not entirely prevented) by a valve 219 that is located within the passageway 217 extending through the energy delivery member 207 and which is similar (if not identical to) the valves 151 (FIG. 12A), 179 (FIGS. 14B, 14C). In alternate embodiments of the disclosure, the configuration of the valve 219 may be reversed (inverted) such that the valve 219 is configured to inhibit (if not entirely prevent) antegrade blood flow (e.g., in the distal direction indicated by the arrow 2) through the energy delivery members 205, 207 and/or that the valve 219 may instead be located within the passageway 215 extending through the energy delivery member 205.

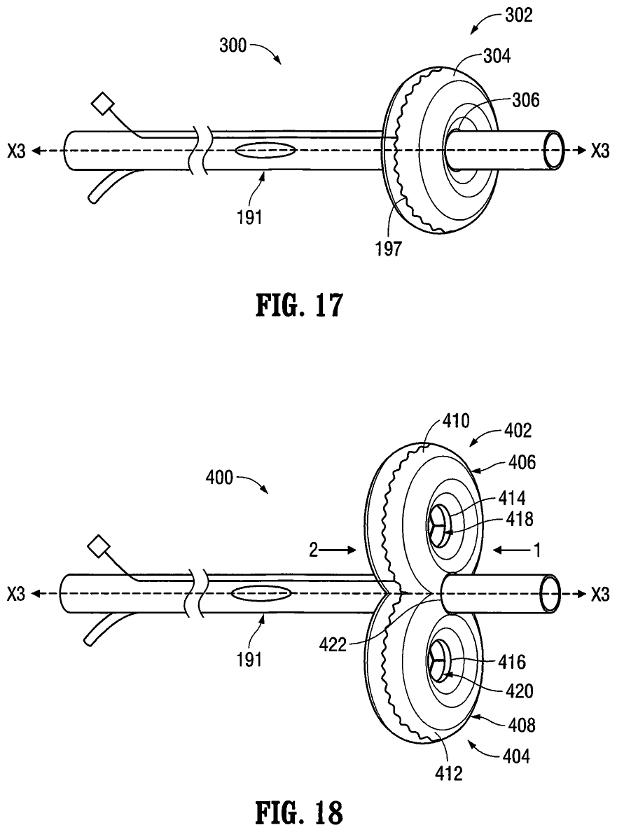

FIG. 17 illustrates another embodiment of the (bypass) catheter, which is identified by the reference character 300. The catheter 300 is substantially similar to the catheter 190 (FIG. 14) and, accordingly, will only be discussed with respect to any differences therefrom in the interest of brevity. As such, identical reference characters will be utilized to refer to elements, structures, features, etc., common to the catheters 190, 300.

In contrast to the catheter 190, the catheter 300 includes an energy delivery member 302 with a toroidal (or generally toroidal, torus configuration) that defines an outer surface 304 with a continuous curvature. The energy delivery member 302 includes (defines) a channel 306 that is configured to receive the catheter body 191 such that the catheter body 191 extends therethrough. In the particular embodiment illustrated, the energy delivery member 302 is centrally positioned about (concentrically mounted to) the catheter body 191. It is also envisioned, however, that the energy delivery member 302 may be eccentrically positioned (e.g., such that the channel 306 is radially offset from the longitudinal axis X3 defined by the catheter body 191).

In the illustrated embodiment, the energy delivery member 302 includes a single energy emitter 197, which is positioned on (or adjacent to) the outer surface 304 of delivery member 302. It should be appreciated, however, that the number of energy emitters 197 included on the energy delivery member 302 may be varied in alternate embodiments without departing from the scope of the present disclosure. Additionally, while illustrated as entirely circumscribing the energy delivery member 302 in FIG. 17, it should be appreciated that the particular configuration of the energy emitter(s) 197 may be varied in alternate embodiments without departing from the scope of the present disclosure. For example, embodiments in which the energy emitter(s) 197 only partially circumscribe the energy delivery member 302 are also envisioned herein.

FIG. 18 illustrates another embodiment of the (bypass) catheter, which is identified by the reference character 400. The catheter 400 is substantially similar to the catheters 190 (FIG. 14), 300 (FIG. 17) and, accordingly, will only be discussed with respect to any differences therefrom in the interest of brevity. As such, identical reference characters will be utilized to refer, to elements, structures, features, etc., common to the catheters 190, 300, 400.

The catheter 400 includes a pair of energy delivery members 402, 404 that are oriented in adjacent (e.g., side-by-side, stacked) relation so as to define a lemniscate (or generally lemniscate) configuration. Although illustrated as being conjoined in the particular embodiment illustrated in FIG. 18, it should be appreciated that the energy delivery members 402, 404 may be configured as separate (discrete) structures in alternate embodiments without departing from the scope of the present disclosure.

Each of the energy delivery members 402, 404 includes a toroidal (or generally toroidal, torus configuration) similar (if not identical) to that discussed in connection with the energy delivery member 302 (FIG. 17). More specifically, the energy delivery members 402, 404 include bodies 406, 408 that respectively define outer surfaces 410, 412 and include passageways 414, 416 that extend therethrough in parallel (or generally) parallel relation to the longitudinal axis X3 defined by the catheter body 191. In certain embodiments, such as that illustrated in FIG. 18, the energy delivery members 402, 404 may include valves 418, 420 that are respectively located within the passageways 414, 416, which are similar (if not identical to) the aforedescribed valves 151 (FIG. 12A), 151B (FIG. 12B), 179 (FIG. 14B). The valves 418, 420 may be configured to inhibit (if not entirely prevent) retrograde blood flow (e.g., backflow) through the respective energy delivery members 402, 404 (e.g., in the proximal direction indicated by the arrow 1) or that the configuration of the valves 418, 420 may be reversed (inverted) such that the valves 418, 420 are configured to inhibit (if not entirely prevent) antegrade blood flow (e.g., in the distal direction indicated by the arrow 2) through the energy delivery members 402, 404.

As seen in FIG. 18, each of the energy delivery members 402, 404 is eccentrically supported by the catheter body 191 (e.g., such that the passageways 414, 416 and the valves 418, 420 are radially offset from the longitudinal axis X3). To facilitate connection (mounting) to the catheter body 191, the energy delivery members 402, 404 collectively define a channel 422 that is configured to receive the catheter body 191 such that the catheter body 191 extends therethrough.

In the illustrated embodiment, the catheter 400 includes a single energy emitter 197, which is positioned on (adjacent to) the respective outer surfaces 410, 412 of the bodies 406, 408 of the energy delivery members 402, 404. It should be appreciated, however, that the number of energy emitters 197 may be varied in alternate embodiments without departing from the scope of the present disclosure. For example, each energy delivery member 402, 404 may support (include) a separate (discrete) energy emitter 197. Additionally, while illustrated as entirely circumscribing the outer surfaces 410, 412 in FIG. 18, it should be appreciated that the particular configuration of the energy emitter(s) 197 may also be varied in alternate embodiments. For example, embodiments in which the energy emitter(s) 197 only partially circumscribe the outer surfaces 410, 412 are also envisioned herein.

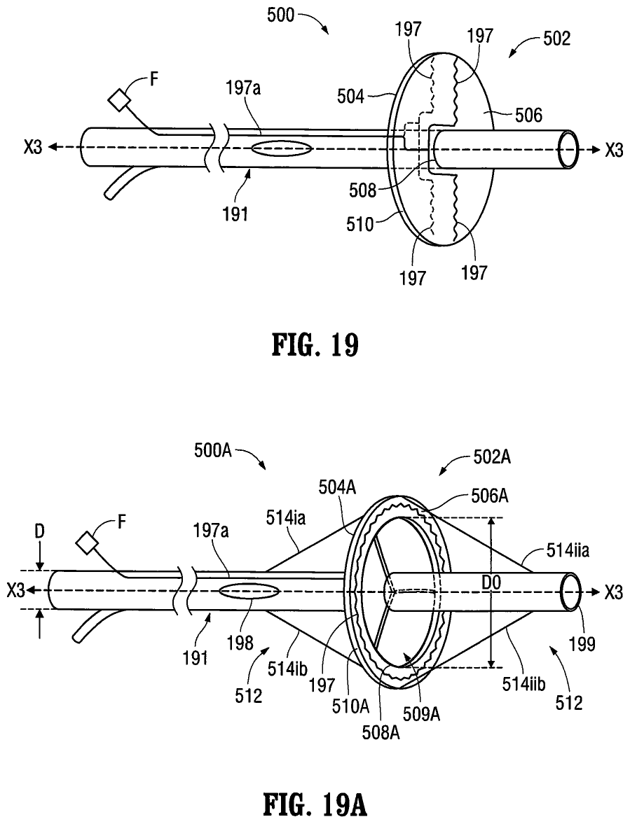

With reference now to FIG. 19, another embodiment of the (bypass) catheter will be discussed, which is identified by the reference character 500. The catheter 500 includes an energy delivery member 502 and is substantially similar to the catheters 190 (FIG. 14), 300 (FIG. 17), 400 (FIG. 18), but for the configuration of the energy delivery member 502. Accordingly, the catheter 500 will only be discussed with respect to any differences from the catheters 190, 300, 400 in the interest of brevity and identical reference characters will be utilized to refer to elements, structures, features, etc., common to the catheters 190, 300, 400, 500.

The energy delivery member 502 is supported by (e.g., mounted on or otherwise secured to) the catheter body 191 such that the energy delivery member 502 extends radially outward therefrom. In contrast to the aforedescribed energy delivery members (e.g., the energy delivery member 155 (FIGS. 12, 12A), the energy delivery member 195 (FIGS. 14-14E), the energy delivery member 175 (FIGS. 15-15, 15A), etc.), the energy delivery member 502 is non-expandable, which allows the configuration of the catheter 500 to be simplified by omitting any need for inflation lumens, thereby allowing for reductions in the overall size and/or cost of the catheter body 191. While the catheter 500 is illustrated as including a single energy delivery member 502, it should be appreciated that the number of energy delivery members 502 may be increased in alternate embodiments of the disclosure, as discussed in further detail below. For example, it is envisioned that the catheter 500 may include two energy delivery members 502, three energy delivery members 502, etc. Additionally, while the energy delivery member 502 is illustrated as being fixedly supported on (connected to) the catheter body 191 in the particular embodiment shown in FIG. 19, in alternate embodiments, the energy delivery member 502 may be movably (e.g., slidably) supported on (connected to) the catheter body 191, as discussed in further detail below.

The energy delivery member 502 includes a deformable (e.g., flexible) material that allows for resilient reconfiguration (e.g., bending, compression, etc.) of the energy delivery member 502 during insertion and removal of the catheter 500. More specifically, the energy delivery member 502 includes a disc-like configuration defining an annular (or generally annular) transverse cross-sectional configuration (e.g., diameter) and respective proximal and distal end faces 504, 506. Although illustrated as being circular (or generally circular) in the particular embodiment seen in FIG. 19, it should be appreciated that alternate configurations for the energy delivery member 502 are also contemplated herein. For example, it is envisioned that the energy delivery member 502 may include a transverse cross-sectional configuration that is elliptical (or generally elliptical), lemniscate (or generally lemniscate), etc. Additionally, while the end faces 504, 506 of the energy delivery member 502 are illustrated as being planar (or generally planar) in the particular embodiment of the disclosure illustrated in FIG. 19, it should be appreciated that the specific configuration of the end faces 504, 506 may vary in alternate embodiments of the disclosure. For example, embodiments in which either or both of the end faces 504, 506 may include an arcuate (e.g., concave or convex) configuration are also contemplated herein.

In the particular embodiment illustrated, the energy delivery member 502 is centrally positioned (concentrically mounted) relative to the longitudinal axis X3 defined by the catheter body 191. More specifically, the energy delivery member 502 includes a passageway 508 that is configured to receive the catheter body 191 such that the catheter body 191 extends through the energy delivery member 502 (in both the proximal and distal directions). It is also envisioned, however, that the energy delivery member 502 may be eccentrically positioned (e.g., such that a center of the energy delivery member 502 is radially offset from the longitudinal axis X3), as described in further detail below.

The energy delivery member 502 includes (supports) one or more energy emitters 197 that is/are in communication with the external energy source F via the connector 197a and which is/are similar or identical to the aforedescribed energy emitters 157, 187, 197, etc. The energy emitter(s) 197 are supported by the energy delivery member 502 such that they are positioned on (adjacent to) the end face 504 and/or the end face 506 and such that they extend around (about) the passageway 508 in a non-linear (e.g., tortuous) configuration. In the particular embodiment illustrated, for example, the energy emitters 197 are positioned on (adjacent to) outer surfaces of the end faces 504, 506 and are directed in opposite (or generally opposite) directions. As discussed in connection with the preceding embodiments of the disclosure, however, it is envisioned that the energy emitter(s) 197 may be arranged in a variety of arrays and spaced in any manner suitable for the intended purpose of communicating energy to the target tissue to facilitate treatment in the manner described herein. For example, the energy emitter(s) 197 may extend about a periphery 510 (e.g., a circumference or outermost radial surface) of the energy delivery member 502, either partially or entirely (e.g., depending upon the particular configuration, location, etc., of the target tissue being treated), and/or that the energy emitter(s) 197 may extend onto the end faces 504, 506 from within the energy delivery member 502 (e.g., in a linear or generally linear configuration). The energy emitter(s) 197 may alternatively be located internally (within) the energy delivery member 502 (e.g., the energy emitter(s) 197 may be embedded within the material used in construction of the energy delivery member 502 such that the energy emitter(s) 197 are positioned radially inward of the end faces 504, 506). In such embodiments, the energy emitter(s) 197 may be exposed via one or more openings (e.g., windows) formed in the end faces 504, 506. In alternate embodiments, the energy emitter(s) can be positioned on only one of the end faces 504, 506.

FIG. 19A illustrates another embodiment of the (bypass) catheter, which is identified by the reference character 500A. The catheter 500A includes an energy delivery member 502A and is substantially similar to the catheter 500 (FIG. 19), but for the energy delivery member 502A, and, accordingly, will only be discussed with respect to any differences therefrom in the interest of brevity. As such, identical reference characters will be utilized to refer to elements, structures, features, etc., common to the catheters 500, 500A.

The energy delivery member 502A defines respective proximal and distal end faces 504A, 506A and includes a passageway 508A and a valve 509A. The valve 509A is similar (if not identical to) the aforedescribed valves 151 (FIGS. 12, 12A), 179 (FIG. 15) and is located (supported) within the passageway 508A such that the catheter body 191 extends therethrough (in both the proximal and distal directions). Although illustrated as being centrally positioned in the particular embodiment seen in FIG. 19A, it is also envisioned that the passageway 508A and the valve 509A may be eccentrically positioned (e.g., such that the passageway 508A and the valve 509A are radially offset from a center of the energy delivery member 502A and the longitudinal axis X3 defined by the catheter body 191).

In contrast to the energy delivery member 502 included on the catheter 500, in which the passageway 508 defines a transverse cross-sectional dimension (e.g., a diameter) substantially approximating that defined by the catheter body 191, the passageway 508A extending through the energy delivery member 502A defines a transverse cross-sectional dimension (e.g., a diameter) D0 substantially exceeding that defined by the catheter body 191, which is identified by the reference character D. For example, it is envisioned that transverse cross-sectional dimension D0 may lie substantially within the range approximately 125% to approximately 500% of the transverse cross-sectional dimension D.

The energy delivery member 502A includes (supports) one or more of the aforementioned energy emitters 197, which, in the illustrated embodiment, are positioned on (adjacent to) the end faces 504A, 506A and extend concentrically around (about) the passageway 508A. Although illustrated as including a single energy emitter 197 on each of the end faces 504A, 506A in the particular embodiment seen in FIG. 19A, it is envisioned that the number of energy emitters 197 may be increased or decreased in alternate embodiments. For example, embodiments including one or more energy emitters 197 solely on the end face 506A or solely on end face 504a are also contemplated herein, as are embodiments including two or more energy emitters 197 on each of the end faces 504A, 504B and embodiments in which one of the end faces 504A, 504B may be devoid of any energy emitters 197. As discussed in connection with the preceding embodiments, the energy emitter(s) 197 may be arranged in a variety of arrays and spaced in any manner suitable for the intended purpose of communicating energy to the target tissue to facilitate treatment in the manner described herein. For example, it is envisioned that the energy emitter(s) 197 may extend along a periphery 510A (e.g., a circumference or outermost radial surface) of the energy delivery member 502A.

In certain embodiments, such as that illustrated in FIG. 19A, the catheter 500A may include one or more braces 512 (e.g., struts 514) that are configured to support and/or stabilize the energy delivery member 502A. For example, in FIG. 19A, the catheter 500A includes a pair of (first) proximal struts 514ia, 514ib and a pair of (second) distal struts 514iia, 514iib that extend between (e.g., are secured or otherwise connected to) the catheter body 191 and the energy delivery member 502A. It should be appreciated, however, that the particular number and/or the configuration of the struts 514 may be altered in various embodiments. For example, an embodiment including a single proximal strut 514i and a single distal strut 514ii is also contemplated herein, as are embodiments including three or more proximal struts 514i and three or more distal struts 514ii. Additionally, while the struts 514 are illustrated as being secured (connected) to the periphery 510A of the energy delivery member 502A, the struts 514 instead or in addition can be secured (connected) to the end faces 504A, 506A.

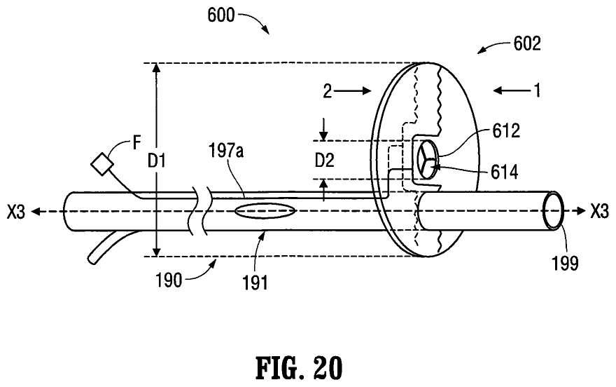

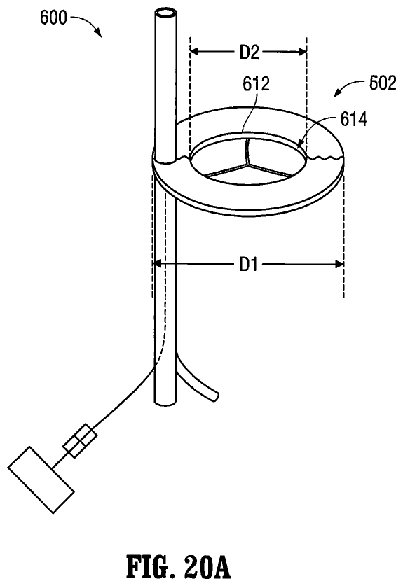

FIG. 20 illustrates another embodiment of the (bypass) catheter 500, which is identified by the reference character 600. The catheter 600 is substantially similar to the catheter 500 (FIG. 19) and, accordingly, will only be discussed with respect to any differences therefrom in the interest of brevity. As such, identical reference characters will be utilized to refer to elements, structures, features, etc., common to the catheters 500, 600.

The catheter 600 includes an alternate embodiment of the energy delivery member 502, which is identified by the reference character 602. The energy delivery member 602 is substantially similar to the energy delivery member 502, but for the inclusion of an aperture 612 (or other such opening), which extends therethrough. The aperture 612 includes (or otherwise supports) a valve 614, which is similar (if not identical to) the aforedescribed valves 151 (FIGS. 12, 12A), 179 (FIG. 15). While the aperture 612 and, thus, the valve 614 are illustrated as being concentrically positioned (e.g., centered or generally centered on the energy delivery member 602), it should be appreciated that the aperture 612 and the valve 614 may be eccentrically positioned in alternate embodiments (e.g., such that the aperture 612 and the valve 614 are radially offset from a center of the energy delivery member 602).

In the embodiment of in FIG. 20, whereas the energy delivery member 602 defines a (first) transverse cross-sectional dimension (e.g., a diameter) D1, the aperture 612 (and the valve 614) defines a (second) transverse cross-sectional dimension (e.g., a diameter) D2 that lies substantially within the range of approximately 10% to approximately 30% of the transverse cross-sectional dimension D1. It should be appreciated, however, that the ratio between the transverse cross-sectional dimensions D1, D2 may be altered in various embodiments. For example, FIG. 20a illustrates an embodiment of the catheter 600 in which the transverse cross-sectional dimension D2 defined by the aperture 612 (and the valve 614) lies substantially within the range of approximately 40% to approximately 90% of the transverse cross-sectional dimension D1. (Otherwise, the device of FIG. 20A is the same as FIG. 20 and can optionally include a side hole for blood inflow).

With reference again to FIG. 20, the energy delivery member 602 is eccentrically mounted on the catheter body 191, whereby the energy delivery member 602, the aperture 612, and the valve 614 are each radially offset from (out of alignment with) the longitudinal axis X3 defined by the catheter body 191 (e.g., such that a majority (more than 50%) of the energy delivery member 602, the aperture 612, and the valve 614 are offset to one side of the longitudinal axis X3). Embodiments are also envisioned, however, in which the energy delivery member 602 may be concentrically positioned about the catheter body 191 while the aperture 612 and the valve 614 remain radially offset from the longitudinal axis X3.

The valve 614 is configured to inhibit (if not entirely prevent) retrograde blood flow (e.g., backflow) through the energy delivery member 602 (e.g., in the proximal direction indicated by the arrow 1). It is also envisioned, however, that the configuration of the valve 614 maybe reversed (inverted) such that the valve 614 is configured to inhibit (if not entirely prevent) antegrade blood flow through the energy delivery member 602 (e.g., in the distal direction indicated by the arrow 2).

While the catheter 600 is illustrated as including a single energy delivery member 602 in the particular embodiment shown in FIG. 20, it should be appreciated that the number of energy delivery members 602 may be increased in alternate embodiments. Additionally, while the energy delivery member 602 is illustrated as being fixedly supported on (connected to) the catheter body 191 in the particular embodiment shown in FIG. 20, in alternate embodiments the energy delivery member 602 may be movably (e.g., slidably) supported on (connected to) the catheter body 191, as discussed in further detail below.

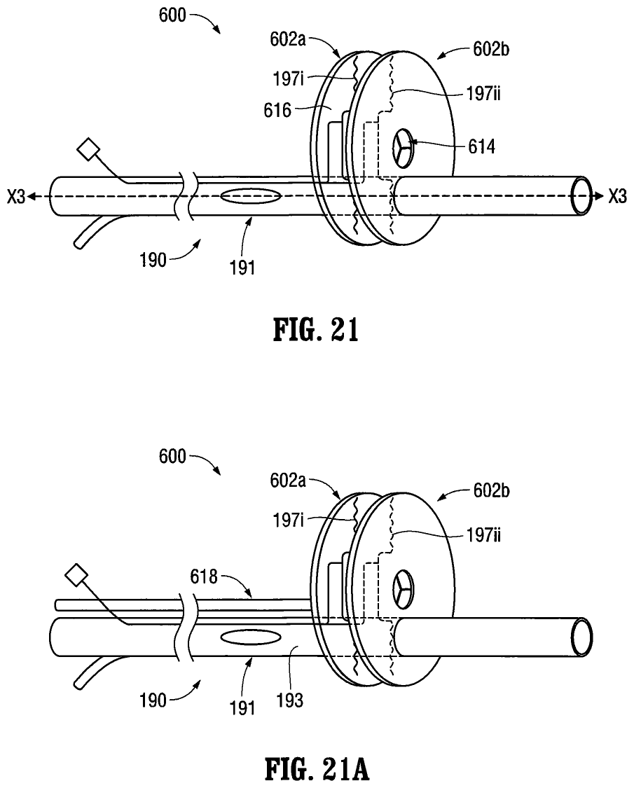

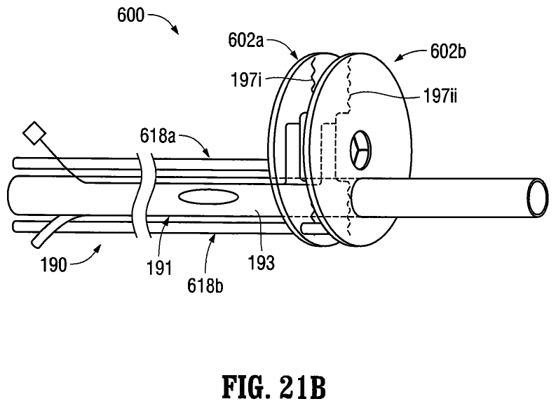

FIG. 21 illustrates an alternate embodiment of the (bypass) catheter 600, which includes a proximal (first) energy delivery member 602a and a second (distal) energy delivery member 602b, each of which is substantially similar (if not identical) to the energy delivery member 602 (FIG. 20). Although illustrated as including a pair of (identical or generally identical) energy delivery members 602, it should be appreciated that the number of energy delivery members 602 may be increased in alternate embodiments, such that the catheter 600 includes three energy delivery members 602, four energy delivery members 602, etc. Embodiments are also envisioned in which the energy delivery members 602a, 602b may be dissimilar in size, shape, flow restriction, etc. For example, embodiments are envisioned in which only one of the energy delivery members 602 (e.g., the energy delivery member 602b) includes the valve 614.

The energy delivery members 602a, 602b are spaced axially from each other along the longitudinal axis X3 defined by the catheter body 191 so as to define a receiving space (chamber) 616. The receiving space 616 is configured to accommodate (receive) the target tissue (e.g., a patient's (calcified) cardiac valve C (FIG. 14D) such that the energy delivery members 602a, 602b are located on opposite sides of the target tissue. Energy delivery members 602a, 602b may be configured and/or located along the catheter body 191 such that either or both of the energy delivery members 602a, 602b contacts the target tissue during the course of the surgical (e.g., lithotripsy) procedure. When so located, the energy emitters 197 are positioned in adjacent (or generally adjacent) relation to the target tissue (e.g., such that the energy emitters 197 are in contact therewith).

In the particular embodiment illustrated in FIG. 21, the energy delivery member 602a includes an energy emitter 197i, which is oriented towards (faces) the energy delivery member 602b, and the energy delivery member 602b includes an energy emitter 197ii, which is oriented towards (faces) the energy delivery member 602a, whereby the energy emitters 197i, 197ii face in opposite (or generally opposite) directions. As discussed in connection with the preceding embodiments, the energy emitter(s) 197 may be arranged in a variety of arrays and spaced in any manner suitable for the intended purpose of communicating energy to the target tissue to facilitate treatment in the manner described herein.

While the energy delivery members 602a, 602b are illustrated as being fixedly supported on (connected to) the catheter body 191 in the embodiment shown in FIG. 21, in alternate embodiments, one or more of the energy delivery members 602a, 602b may be movably (e.g. slidably) supported on (connected to) the catheter body 191 to allow the configuration of the receiving space 616 to be varied (e.g., depending upon the particular configuration, location, etc., of the target tissue being treated) to adjust the distance from tissue, degree of contact, pressure against tissue, etc. by the energy delivery members 602a, 602b. For example, FIG. 21A illustrates an embodiment in which the energy delivery member 602b is fixedly supported on (connected to) the catheter body 191 while the energy delivery member 602a is movable in relation thereto via a pusher 618. In such embodiments, the pusher 618 may be located externally of the catheter body 191, as illustrated in FIG. 21A. The pusher 618 may extend through (or may be embedded within) the wall of the catheter body 191, may extend through the (main, working) lumen 193 or the catheter body 191 may include a separate (discrete) lumen that is configured to receive the pusher 618. Embodiments in which each of the energy delivery members 602a, 602b may be movable in relation to the catheter body 191 (e.g., via corresponding pushers 618a, 618b) are also contemplated herein, as illustrated in FIG. 21B. Pusher 618a effects axial movement of delivery member 602a and pusher 618b effects axial movement of delivery member 602b to enable the clinician to selectively adjust the distance between the energy delivery members 602a, 602b to adjust the distance relative to the target tissue and the extent and force of contact with target tissue located in the gap between energy delivery members 602a, 602b.

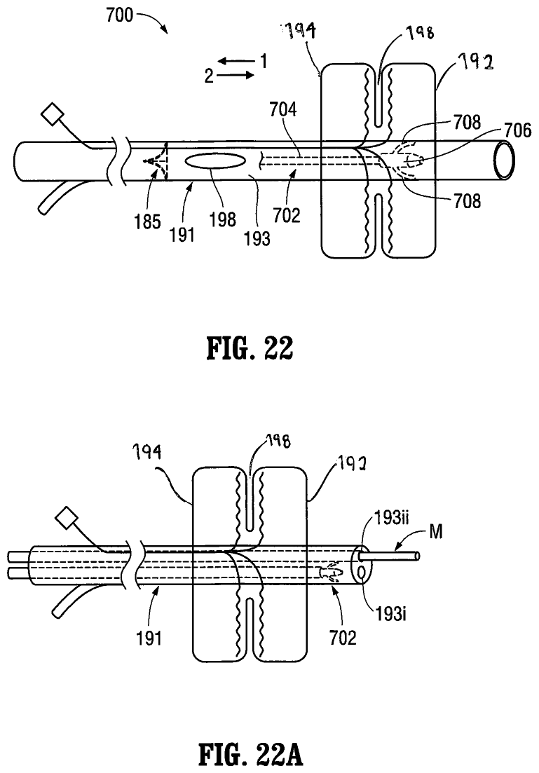

FIG. 22 illustrates another embodiment of the (bypass) catheter, which is identified by the reference character 700. The catheter 700 is substantially similar to the catheter 190 (FIGS. 14, 14A) and, accordingly, will only be discussed with respect to any differences therefrom in the interest of brevity. As such, identical reference characters will be utilized to refer to elements, structures, features, etc., common to the catheters 190, 700.

The catheter 700 includes a flow circulator 702 that is rotatably located (positioned, secured) within the catheter body 191 so as to direct blood flow therethrough. The flow circulator 702 may be configured for unidirectional or bidirectional rotation to direct blood flow through the catheter body 191 in the proximal direction e.g., through the valve, (indicated by the arrow 1) and/or or the distal direction (indicated by arrow 2), depending upon the particular direction of rotation of the flow circulator 702. While the flow circulator 702 is illustrated as an impeller 704 including a shaft 706 that supports a head 706 with a plurality of vanes (blades) 708 that extend radially outward therefrom in the particular embodiment seen in FIG. 22, it should be appreciated that the other configurations of the flow circulator 702 are also contemplated and that the flow circulator 702 may include (or may be configured as) any member or structure suitable for the intended purpose of directing blood flow through the catheter body 191 in the manner described herein. Further details regarding the flow circulator 702 (and embodiments thereof) are provided in U.S. patent application Ser. No. 16/881,727, the entire contents of which are hereby incorporated by reference. The motor for actuating the flow circulator can be external to the catheter or alternatively positioned within the catheter and can be powered by a battery or external plug.

While the flow circulator 702 is illustrated as extending within (through) the (main, working) lumen 193 in the embodiment of FIG. 22, the catheter body 191 may alternatively include separate lumens for the flow circulator 702 and the supplemental medical device M. For example, FIG. 22A illustrates an embodiment in which the catheter body 191 includes a (first) lumen 193i that is configured to receive (or otherwise accommodate) the flow circulator 702 and a (second) lumen 193ii that extends in parallel (or generally parallel) relation to the lumen 193i and which is configured to receive (or otherwise accommodate) the supplemental medical device M. Embodiments of the disclosure are also envisioned in which the flow circulator 702 and the supplemental medical device M may be accommodated within the same lumen (e.g., the lumen 193 (FIG. 22). In such embodiments, the catheter body 191 and the flow circulator 702 may be configured such that the flow circulator 702 is rotatable around (about) the supplemental medical device M.

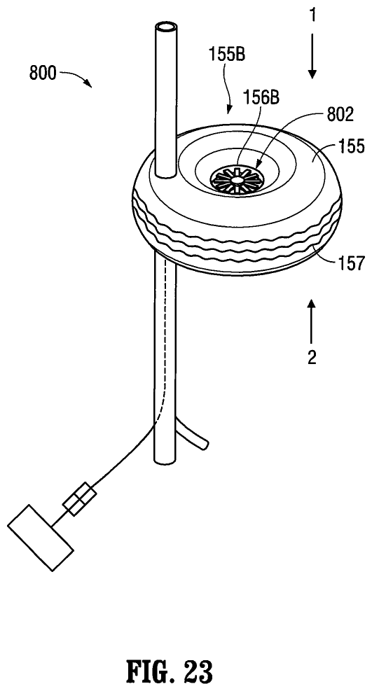

FIG. 23 illustrates another embodiment of the (bypass) catheter, which is identified by the reference character 800. The catheter 800 is substantially similar to the catheter 140 (of FIG. 12B) in that the energy delivery member is the same configuration. However, it differs from FIG.12B in that it includes a flow circulator 802 which enhances flow in the distal and/or proximal direction in the same manner as flow circulator 702 of FIGS. 22 and 22A. Accordingly, catheter 800 will only be discussed with respect to any differences therefrom in the interest of brevity and identical reference characters will be utilized to refer to elements, structures, features, etc., common to the catheter of FIG. 12B.

In contrast to the catheter 700, in which the flow circulator 702 is located (positioned, secured) within the catheter body 191, the catheter 800 includes a flow circulator 802 that is supported by the energy delivery member 155B. More specifically, the flow circulator 802 is included (provided) within the passageway 156B of delivery member 155B so as to direct blood flow though the energy delivery member 155B and replaces the aforementioned valve 151B of FIG. 12B. As discussed in connection with the catheter 700, depending upon the particular direction of rotation, the flow circulator 802 may direct blood flow through the energy delivery member 155B in the proximal direction (indicated by the arrow 1) or the distal direction (indicated by arrow 2). Note the flow circulator 802 can be placed within openings/passageways of other energy delivery members disclosed herein.

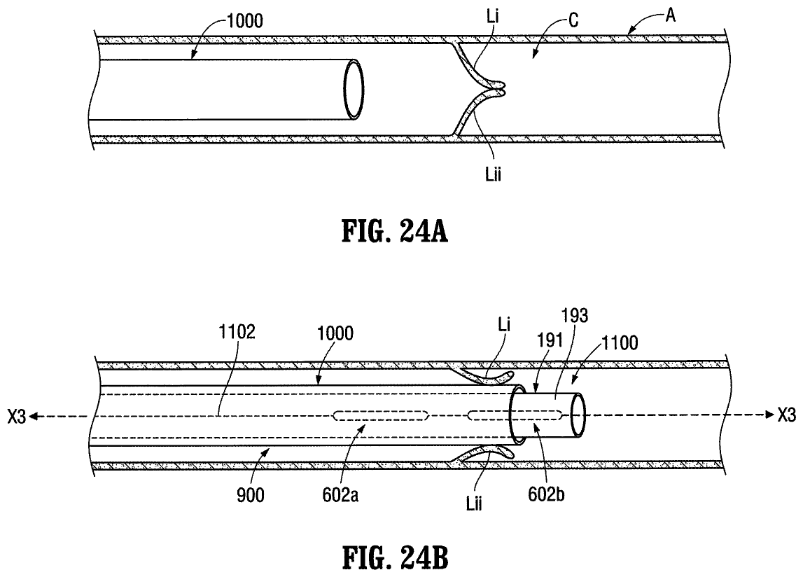

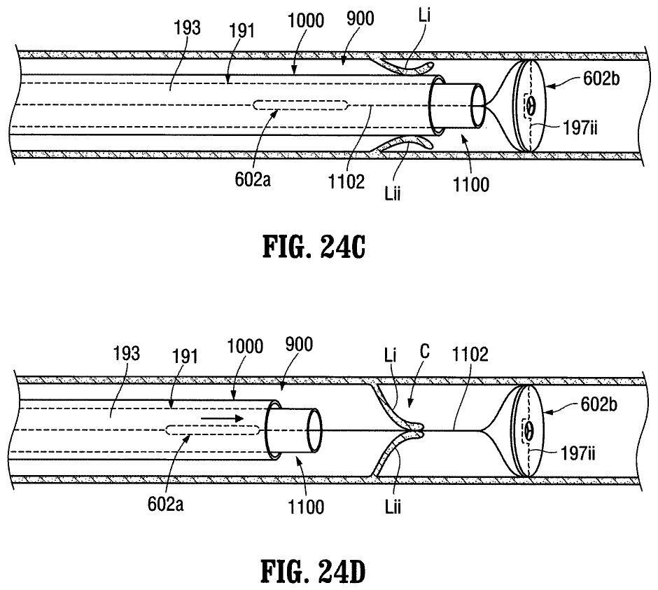

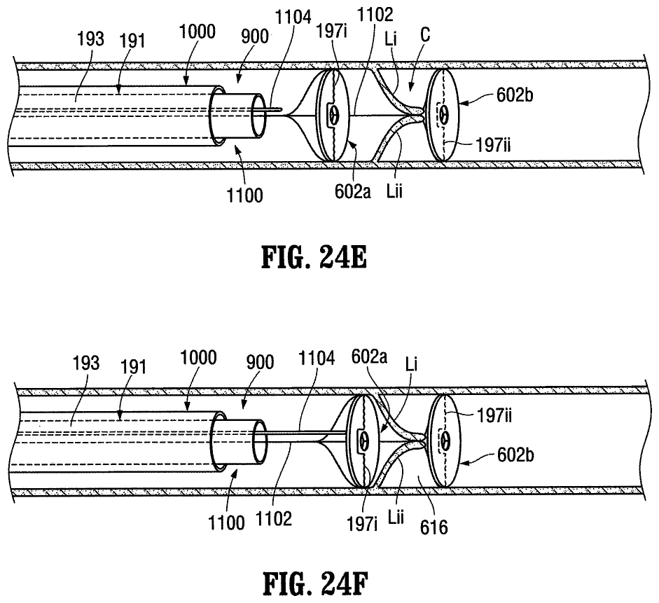

With reference now to FIGS. 24A-F, a method of performing a surgical procedure (e.g., a lithotripsy procedure) will be discussed in connection with a surgical system 900, which includes a delivery (outer) catheter 1000 and another embodiment of the presently disclosed (bypass) catheter, which is identified by the reference character 1100. The (inner) catheter 1100 is substantially similar to the catheter 600 (FIGS. 20-21B) and, accordingly, will only be discussed with respect to any differences therefrom in the interest of brevity. As such, identical reference characters will be utilized to refer to elements, structures, features, etc., common to the catheters 600, 1100. Note the other catheters disclosed herein can be used for performing lithotripsy as well as other procedures.

The delivery catheter 1000 is configured to receive the catheter 1100 to facilitate placement thereof within the access vessel A (see FIG. 14D as well) in a manner that allows for access to the target tissue, which, in the particular procedure shown, is illustrated as a patient's cardiac valve C. While the delivery catheter 1000 and the catheter 1100 are each illustrated as including a circular (or generally circular) transverse cross-sectional configuration (e.g., diameter), alternative configurations are also contemplated herein. For example, it is envisioned that the delivery catheter 1000 and the catheter 1100 may include corresponding non-circular transverse cross-sectional configurations (e.g., square, rectangular, hexagonal, octagonal, pentagonal, a “house” silhouette, oval, elliptical, star-shaped, etc.) so as to inhibit (if not entirely prevent) relative rotation between the delivery catheter 1000 and the catheter 1100. In the context of a star-shaped transverse cross-sectional configuration, any style of star may be used including, for example, a six-pointed star, a “Star of David,” etc. In certain embodiments, it is also envisioned that the delivery catheter 1000 and the catheter 1100 may include different transverse cross-sectional configurations.

In contrast to the catheter 600 (FIGS. 20-21B), in which the energy delivery members 602a, 602b are supported externally of the catheter body 191, in the context of the catheter 1100, the energy delivery members 602a, 602b are supported on (engaged by) a carrier 1102 (e.g., a rod, a wire, etc.), which is configured for insertion into the catheter body 191 (e.g., via the lumen 193) such that the catheter body 191 and the carrier 1102 are axially movable in relation to each other (e.g., along the longitudinal axis X3).

During the course of the surgical procedure, the delivery catheter 1000 is positioned within the access vessel A (FIG. 24A) and is advanced toward the target tissue. As seen in FIG. 24B, in the particular procedure shown, the delivery catheter 1000 is advanced through the cardiac valve C, thereby separating the leaflets Li, Lii. The catheter 1100 is then advanced through the cardiac valve C and the energy delivery member 602b is deployed by causing relative axial movement between the energy delivery member 602b and the catheter body 191. For example, deployment of the energy delivery member 602b may be accomplished by advancing the carrier 1102 distally relative to the catheter body 191 (and the delivery catheter 1000) and/or by retracting (withdrawing) the catheter body 191 (and the delivery catheter 1000) relative to the carrier 1102. During deployment, as the energy delivery member 602b exits the catheter body 191, the energy delivery member 602b is (automatically) reconfigured from a collapsed (first, compressed) configuration (FIG. 24B) to an expanded (second) configuration (FIG. 24C), which is facilitated by the flexible, resilient construction of the energy delivery member 602b mentioned above. The delivery catheter 1000 and the catheter body 191 are then moved proximally (retracted) such that they are located proximally of the target tissue, as seen in FIG. 24D, and the carrier 1102 is moved proximally (retracted) such that the energy delivery member 602b contacts the cardiac valve C (or is positioned in close proximity thereto), as seen in FIG. 24E. In certain methods of use, it is envisioned that proximal movement of the carrier 1102 may be accomplished in concert with the delivery catheter 1000 and the catheter 1100.

Following positioning of the energy delivery member 602b in the manner illustrated in FIG. 24E, the energy delivery member 602a is deployed proximally of the cardiac valve C by causing relative axial movement between the carrier 1102 and the catheter body 191 (and the delivery catheter 1000) in a similar (if not identical) manner to that discussed above in connection with deployment of the energy delivery member 602b. During deployment, like the energy delivery member 602b, as the energy delivery member 602a exits the catheter body 191, the energy delivery member 602a is (automatically) reconfigured from a collapsed (first, compressed) configuration (FIG. 24B-24D) to an expanded (second) configuration (FIG. 24E), which is again facilitated by the flexible, resilient construction of the energy delivery member 602a mentioned above. The energy delivery member 602a is then positioned such that the energy delivery member 602a contacts the cardiac valve C (or is positioned in close proximity thereto), as seen in FIG. 24F, such that the cardiac valve C (e.g., the leaflets Li, Lii) are located between the energy delivery members 602a, 602b (e.g., within the space 616) between the energy delivery members 602a, 602b.

In the particular embodiment of the disclosure illustrated in FIGS. 24A-F, the energy delivery member 602a is movably (e.g., slidably) supported on (engaged by) the carrier 1102, which allows for relative axial movement between the energy delivery member 602a and the carrier 1102 (e.g., along the longitudinal axis X3). To facilitate such movement, it is envisioned that the catheter 1100 may include (or may be used with) a pusher 1104 (FIGS. 24E, 24F), which is substantially similar (if not identical) to the aforedescribed pusher 618 (FIG. 21A). More specifically, force is applied to the pusher 1104 to thereby advance the energy delivery member 602a distally towards the cardiac valve C until the energy delivery member 602a is positioned as illustrated in FIG. 24F. It is envisioned that the pusher 1104, like pusher 618 (and 618a, 618b) may be either fixedly connected to the energy delivery member 602a or, alternatively, that the pusher 1104 and the energy delivery member 602a may be selectively engageable, as illustrated in the FIGS. 24E, 24F, which allows for selective engagement between the pusher 1104 and the energy delivery member 602b as well (e.g., to adjust the position of the energy delivery member 602b). The pushers 1104, 618, etc. may also be configured to enable the energy delivery members to be pulled/moved in a proximal direction.

As discussed above in connection with the catheter 600 (FIG. 21A), the pusher 1104 may be located externally of the catheter body 191 or may extend through (or may be embedded within) the catheter body 191 or may extend through the (main, working) lumen 193 or catheter body 191 may include a separate (discrete) lumen that is configured to receive the pusher 1104.

Following location of the target tissue between the energy delivery members 602a, 602b in the manner illustrated in FIG. 24F, energy is communicated to the target tissue through the energy emitters 197i, 197ii respectively included on the energy delivery members 602a, 602b (in the same manner as discussed above) to thereby treat the target tissue (e.g., soften any calcium deposits on the valve leaflets Li, Lii).

It is envisioned that any of the catheters/devices described herein may optionally include one or more steerable segments (zones) that are deflectable via one or more pull wires (or alternatively push wires) that extend (are embedded) within the wall of the catheter such that the catheter is reconfigurable (i.e., deflectable) between a variety of configurations. Additionally (or alternatively), it is envisioned that any of the catheters (or other devices) described herein may (optionally) include an integrated visualization device or system (e.g., a camera or the like) to facilitate imaging during the course of a surgical procedure.

To vary tension on the pull wires, the catheter may include or may be connected to any suitable mechanism including, for example, a wheel, a ratchet, etc., thereby curving (deflecting) the device, as descried in further detail below. The term “steerability”, as used herein, should be understood as referring to an ability to turn, rotate, or otherwise deflect the catheter/device. While the following discussion is provided in connection with the catheter 190 of FIG. 14, it should be appreciated that the following principles may be equally applicable to any of the catheters (or other devices) described herein.

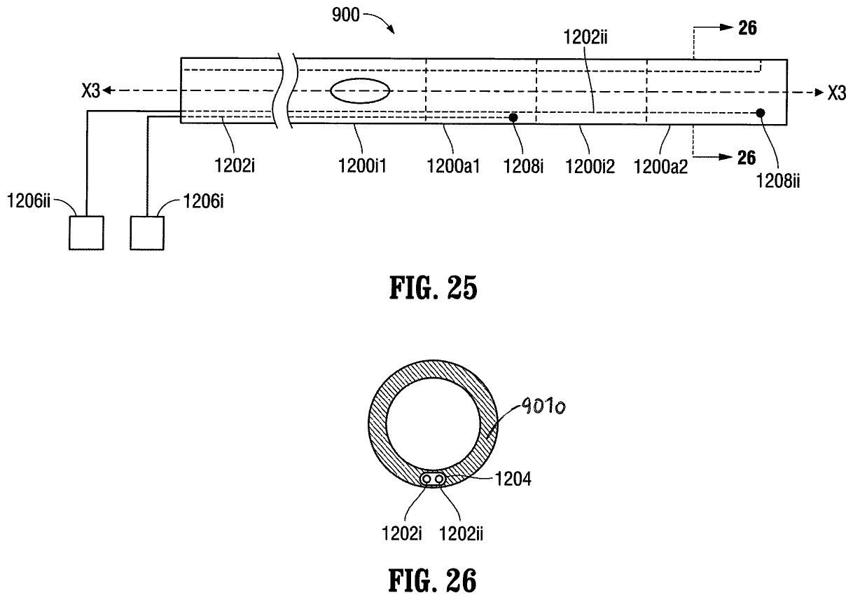

With reference now to FIGS. 25-27, to facilitate articulation (deflection) and reconfiguration, the catheter 900 may include a plurality of segments 1200 and one or more pull wires 1202. For clarity, in FIGS. 25-27, only the catheter body 191 is shown (e.g., the energy delivery member 195, the energy emitters 197, the connector 197a, and the energy source F have been removed from the illustration). The catheter 900 includes a plurality of inactive (passive) segments 1200i and a plurality of active (steerable, deflectable, articulable) segments 1200a that are connected to a plurality of pull wires 1202. The inactive segments 1200i and the active segments 1200a are arranged in a staggered pattern along the longitudinal axis X defined by the catheter body 191 such that the catheter 900 alternates between inactive segments 1200i and active segments 1200a.

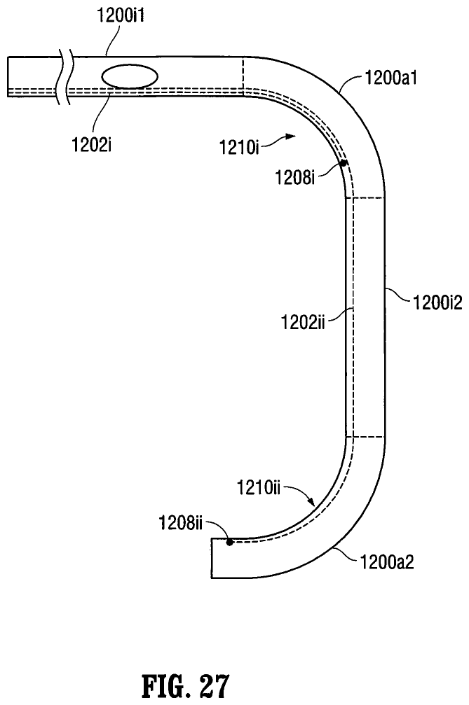

Each active segment 1200a is connected to a corresponding (single) pull wire 1202 that extends through (e.g., within) an outer wall of the catheter 900 (e.g., such that the pull wires 1202 are embedded into the catheter body 191), whereby the number of pull wires 1202 corresponds to the number of active segments 1200a. Upon the application of an axial (pulling) force to each of the pull wires 1202, the corresponding active segment 1200a is deflected (articulated) to thereby reconfigure (actively steer) the catheter 900 between a first (initial, normal) configuration (FIG. 25), in which the catheter 900 includes a (generally) linear configuration, and a second (subsequent, deflected) configuration (FIG. 27), in which the catheter 900 includes a non-linear configuration.

The use of a single pull wire 1202 in connection with each active segment 1200a reduces the requisite number of pull wires 1202, thus reducing complexity in both construction and operation of the catheter 900. It is also envisioned that multiple, independently movable pull wires 1202 may be included in other embodiments. In the particular embodiment illustrated, each pull wire 1202 is received within a corresponding channel 1204 (FIG. 26) that extends through the outer wall 9010 of the catheter 900 in (generally) parallel relation to the longitudinal axis X (e.g., such that the pull wires 1202 are embedded within the catheter 900). In alternate embodiments, the pull wires extend through lumens within the primary center lumen.

To facilitate the application of axial force to the pull wires 1202, in certain embodiments, the catheter 900 may include (or may be connected to) a plurality of corresponding activating mechanisms 1206 (e.g., such that the number of pull wires 1202 corresponds to the number of activating mechanisms 1206). In the particular embodiment illustrated, the catheter 900 includes a (first) activating mechanism 1206i that is connected to the pull wire 1202i and a (second) activating mechanism 1206ii that is connected to the pull wire 1202ii. The activating mechanisms 1206 may include any structure or mechanism suitable for the intended purpose of applying the axial force to the pull wires 1202 required to deflect the catheter 900 as necessary or desired, such as, for example, rotating wheels, pulley systems, or the like. In certain embodiments, the active segments 1200a, the pull wires 1202, and the activating mechanisms 1206 may be configured (and connected) such that each pull wire 1202 may be individually acted upon to deflect (steer) the corresponding segment 1200a in a single direction only. In other embodiments, pull wires 1202 may be provided on various circumferential surfaces of the catheter 900 to facilitate steering of the distal regions of the catheter in various directions. For example, the wire can have a neutral position wherein distal movement deflects the catheter in a first direction and proximal movement deflects the catheter in a second direction. In alternate embodiments, push wires instead of pull wires are provided to effect deflection/articulation.

In the embodiment illustrated, the catheter 900 includes a first inactive segment 1200i1; a first active segment 1200a1 that is located distally of the inactive segment 1200i1; a second inactive segment 1200i2 that is located distally of the active segment 1200a1; and a second active segment 1200a2 that is located distally of the inactive segment 1200i2. Additionally, the catheter 900 includes respective first and second pull wires 1202i, 1202ii that are located within the channel 1204 (FIG. 26). It is also envisioned, however, that the first and second pull wires 1202i, 1202ii may be located within separate channels 1204 (e.g., such that the number of channels 1204 corresponds to the number of pull wires 1202).

The pull wires 1202i, 1202ii are connected to the segments 1200a1, 1200a2 at connection points 1208i, 1208ii (in addition to the activating mechanism 1206i, 1206ii), respectively, so as to facilitate reconfiguration of the catheter 900 between the first configuration (FIG. 25) and the second configuration (FIG. 27). More specifically, upon reconfiguration/deflection of the catheter 900, the active segments 1200ai, 1200aii define respective first and second bends 1210i, 1210ii (FIG. 27), which may be either substantially similar (e.g., identical) or dissimilar depending, for example, upon the particular configuration of the segments 1200a1, 1200a2, the materials of construction used in the catheter 900, the particular requirements of the catheter 900 dictated by the surgical procedure, etc. Although the bends 1210i, 1210ii are each illustrated as being (approximately) equal to 90 degrees in FIG. 27, depending upon the particular configuration of the segments 1200a1, 1200a2, the requirements of the surgical procedure, the particular anatomy of the patient, etc., it is envisioned that the bends 1210i, 1210ii may lie substantially within the range of approximately 0 degrees to approximately 270 degrees. For example, in one particular embodiment, it is envisioned that the segment 1200a1 may be configured such that the bend 1210i lies substantially within the range of approximately 0 degrees to approximately 180 degrees (e.g., approximately 90 degrees to approximately 180 degrees) and that the segment 1200a2 may be configured such that the bend 1210ii lies substantially within the range of approximately 0 degrees to approximately 270 degrees (e.g., approximately 90 degrees to approximately 270 degrees).

In the particular embodiment illustrated, the connection points 1208i, 1208ii are shown as being in (general) angular alignment (e.g., along a circumference of the catheter 900), which facilitates deflection of the segments 1200a1, 1200a2 in similar (e.g., identical) directions, as seen in FIG. 27. It is also envisioned, however, that the connection points 1208i, 1208ii may be angularly offset so as to facilitate deflection of the segments 1200a1, 1200a2 in dissimilar directions. For example, the connection points 1208i, 1208ii may be oriented in (generally) diametric opposition such that the bends 1210i, 1210ii respectively defined by the segments 1200a1, 1200a2 curve in (generally) opposite directions.

It should be appreciated that the number of bendable regions can vary such that there is only one bendable region, or two bendable regions (as in FIG. 27) or more than two bendable regions. The number of pull or push wires would vary to accommodate the number of bendable regions.

Various mechanisms and features can be used to deflect (steer the catheters disclosed herein such as disclosed in PCT application PCT/US22/51599, filed Dec. 2, 2022 and US Publication 2021/0259860. The entire contents of both of these applications are incorporated herein by reference. Wire(s) for applying a torsional (twisting) force to rotate the catheter as disclosed in Publication 2021/0259860 can be utilized with any of the catheters disclosed herein.

As discussed above, the catheters disclosed herein can have circular (or generally circular) or non-circular transverse cross-sections (diameters) and/or circular (or generally circular) or non-circular lumen(s).

The catheters of the present invention are preferably placed in a minimally invasive manner, most often percutaneously, e.g., through the femoral artery or radial artery, and advanced endovascularly (through the vasculature) to the target tissue site, e.g., adjacent the blood clot. The catheters are configured for temporary placement and are removed after the procedure. The catheters can alternatively be left in place over a period of time.

Although generally discussed in the context of a surgical procedure that is performed on an anatomical valve (e.g., a patient's cardiac valve), it should be appreciated that the various embodiments of the catheters described herein may be configured for use during the treatment of other areas and tissues.

Although described in connection with the treatment of blood clots, the catheters disclosed herein can be used to break up or dissolve and/or deliver medications to other regions of the body for performing other surgical procedures wherein immediate reperfusion, continuous and/or controlled blood flow is desired during the surgical procedure. It is ideally adapted for any luminal structures that may have a blockage.

It will be understood that the above particular embodiments are shown and described by way of illustration only. The principles and the features of the present invention may be employed in various and numerous embodiments thereof without departing from the scope and spirit of the disclosure as claimed. The above-described embodiments do not restrict the scope of the disclosure and it should be understood by those skilled in the art that various changes may be made (and equivalents may be substituted) without departing from the true spirit and scope of the present invention. In addition, many modifications may be made to adopt a particular situation, material, composition of matter, process, process step or steps, to the objective spirit and scope of the present invention. All such modifications are intended to be within the scope of the claims appended hereto.

Persons skilled in the art will understand that the elements and features shown or described in connection with one embodiment may be combined with those of another embodiment without departing from the scope of the present invention provided.

Where a range of values is provided, it is understood that each intervening value between the upper and lower limit of that range is encompassed within the present invention.

It must be noted that as used herein and in the appended claims, the singular forms “a”, “and”, and “the” include plural references unless the context clearly dictates otherwise.

Throughout the present disclosure, terms such as “approximately,” “generally,” “substantially,” and the like should be understood to allow for variations in any numerical range or concept with which they are associated. For example, it is intended that the use of terms such as “approximately” and “generally” should be understood to encompass variations on the order of 25% (e.g., to allow for manufacturing tolerances and/or deviations in design).

Although terms such as “first,” “second,” “third,” etc., may be used herein to describe various operations, elements, components, regions, and/or sections, these operations, elements, components, regions, and/or sections should not be limited by the use of these terms in that these terms are used to distinguish one operation, element, component, region, or section from another. Thus, unless expressly stated otherwise, a first operation, element, component, region, or section could be termed a second operation, element, component, region, or section without departing from the scope of the present disclosure.

Each and every claim is incorporated as further disclosure into the specification and represents embodiments of the present disclosure. Also, the phrases “at least one of A, B, and C” and “A and/or B and/or C” should each be interpreted to include only A, only B, only C, or any combination of A, B, and C.

Various combinations of all devices and methods described above may be utilized in the same procedure, sequentially and/or simultaneously.

What is claimed is:

1. A catheter for intraluminal lithotripsy comprising:
a catheter body defining a longitudinal axis and including a first lumen extending therethrough;
an energy delivery member supported by the catheter body such that the energy delivery member extends radially outward therefrom, the energy delivery member including:
a body;
a passageway extending through the body in generally parallel relation to the longitudinal axis;
a valve positioned within the passageway to inhibit blood flow through the energy delivery member; and
an energy emitter configured to communicate energy to target tissue to facilitate treatment thereof; and
a connector extending from the energy emitter to an external energy source to thereby supply energy to the energy emitter.

2. The catheter of claim 1, wherein the first lumen is configured to receive a supplemental medical device.

3. The catheter of claim 1, wherein the energy delivery member is supported by the catheter body such that the passageway is positioned eccentrically relative to the longitudinal axis.

4. The catheter of claim 1, wherein the energy delivery member is expandable.

5. The catheter of claim 4, wherein the energy delivery member includes:
a proximal portion;
a distal portion; and
an intermediate portion positioned between the proximal portion and the distal portion, wherein, upon expansion of the energy delivery member, the proximal portion and the distal portion each define a transverse cross-sectional dimension and the intermediate portion defines a second transverse cross-sectional dimension less than the transverse cross-sectional dimension of the proximal and distal portions such that the energy delivery member includes a waist defining a gap configured to receive the target tissue.

6. The catheter of claim 4, wherein the energy delivery member includes a generally toroidal configuration.

7. The catheter of claim 1, wherein the energy delivery member is generally cylindrical and defines a proximal end face and a distal end face each having a generally planar configuration and the energy emitter is supported adjacent to at least one of the proximal end face and the distal end face.

8. The catheter of claim 1, wherein the energy delivery member includes a deformable material to allow for reconfiguration of the energy delivery member during insertion and removal of the catheter.

9. The catheter of claim 1, further comprising a second energy delivery member, the second energy delivery member being configured as a discrete structure spaced axially from the energy delivery member along the longitudinal axis.

10. The catheter of claim 9, wherein at least one of the energy delivery member and the second energy delivery member is movable along the catheter body.

11. The catheter of claim 1, wherein the catheter is steerable.

12. The catheter of claim 1, wherein the valve opens intermittently.

13. The catheter of claim 1, wherein the valve is configured to treat a heart valve.

14. The catheter of claim 1, wherein a pressure gradient across the valve causes the valve to open.

15. The catheter of claim 14, wherein blood flows through the valve when the valve is open.

16. A catheter for intraluminal lithotripsy comprising:
a catheter body defining a longitudinal axis;
an impeller to direct blood flow;
an energy delivery member extending radially outward from the catheter body, the energy delivery member including:
a body with a generally annular transverse cross-sectional configuration; and
an energy emitter and configured to communicate energy to target tissue to facilitate treatment thereof; and
a connector extending from the energy emitter to an external energy source to thereby supply energy to the energy emitter.

17. The catheter of claim 16, wherein the impeller is positioned within a lumen in the catheter body.

18. The catheter of claim 16, wherein the energy delivery member includes a passageway and the impeller is positioned within the passageway.

19. The catheter of claim 16, wherein the catheter body defines a first lumen configured to receive the impeller and a second lumen.

20. The catheter of claim 19, wherein the second lumen is configured to receive the supplemental medical device.

21. The catheter of claim 16, wherein the energy delivery member is expandable and includes:
a proximal portion;
a distal portion; and
an intermediate portion positioned between the proximal portion and the distal portion, wherein, upon expansion of the energy delivery member, the proximal portion and the distal portion each define a first transverse cross-sectional dimension and the intermediate portion defines a second transverse cross-sectional dimension less than the first transverse cross-sectional dimension such that the energy delivery member includes a waist defining a gap configured to receive the target tissue.

22. The catheter of claim 21, wherein the energy emitter includes at least two spaced apart energy emitting surfaces.

23. The catheter of claim 22, wherein the energy emitting surfaces are configured to press against opposite sides of tissue and are on different sides of the waist.

24. The catheter of claim 23, wherein the energy delivery member is configured to treat a cardiac valve.

25. A method of performing an intraluminal lithotripsy procedure comprising:
a) inserting a catheter including:
a catheter body; and
an energy delivery member extending radially of the catheter body, the energy delivery member including a passageway extending therethrough;
b) intermittently inhibiting blood flow through the energy delivery member using a valve supported within the passageway;
c) positioning the energy delivery member adjacent to target tissue; and
d) applying energy to the energy delivery member to treat the target tissue.

26. The method of claim 25, wherein positioning the energy delivery member adjacent to the target tissue includes expanding the energy delivery member such that the target tissue is received within a gap defined between proximal and distal portions of the energy delivery member.

27. The method of claim 26, wherein positioning the energy delivery member adjacent to the target tissue includes positioning a first energy delivery member distally of the target tissue and positioning a second energy delivery member proximally of the target tissue.

28. The method of claim 25, further comprising directing blood flow through the catheter by rotating an impeller positioned within the catheter body.

29. The method of claim 27, wherein the target tissue is a valve.

30. The method of claim 29, wherein the valve is a cardiac valve.
